(19) United States
(12) Patent Application Publication
(10) Pub. No.: US 2019/0111114 A1
(43) Pub. Date: Apr. 18, 2019

(54) METHODS FOR IDENTIFYING AND TREATING HEMOGLOBINOPATHIES

(71) Applicants: Takahiro MAEDA, Fukuoka (JP); THE BRIGHAM AND WOMEN'S HOSPITAL, INC., Boston, MA (US)

(72) Inventor: Takahiro Maeda, Fukuoka (JP)

(21) Appl. No.: 16/067,733

(22) PCT Filed: Dec. 29, 2016
(86) PCT No.: PCT/US2016/069078

§ 371 Date: Jul. 2, 2018

Related U.S. Application Data
(60) Provisional application No. 62/272,853, filed on Dec. 30, 2015.

(51) Int. Cl. A61K 38/46; C07K 14/47; C12N 9/90; G01N 33/50; G01N 33/68; A61P 7/00; C07K 16/18; C12N 15/10; A61K 38/16; C12N 15/85

(52) U.S. Cl. CPC A61K 38/46; C07K 14/4702; C12N 9/90; G01N 33/5008; G01N 33/6872; A61P 7/00; C12N 2015/8536; C12N 15/1086; A61K 38/162; C12N 15/85; C12Y 306/04012; G01N 2500/02; C12N 2015/859; C07K 16/18

(57) ABSTRACT

The invention relates to method of treating or inhibiting progression of hemoglobinopathy in a subject in need thereof comprising inhibiting interaction between LRF-BTB and CHD protein-LBD.

### INCORPORATION BY REFERENCE

This application claims benefit of and priority to U.S. provisional patent application 62/272,853 filed Dec. 30, 2015, incorporated herein by reference.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

Mention is made of Abstract No. 80611 and Presentation “The LRF/ZBTB7A Transcription Factor is a BCL11A-Independent Repressor of Fetal Hemoglobin” presented at the American Society of Hematology, Dec. 6, 2015, the contents of which are incorporated by reference herein in their entirety. That Abstract and its presentation does not teach or suggest the inventions claimed herein. This mention is to satisfy any requirements of any jurisdiction, e.g., United States, Japan, to disqualify from being prior art any disclosure made within one year or six months, respectively, prior to filing. In this regard, it is also mentioned that the Abstract and its presentation is by the same individual(s) and entity(ies) named as inventor(s) and applicant(s) on the present application, such that there is both disclosure of the Abstract and its presentation on filing of this specification, and identification that the disclosure of the Abstract and its presentation are by the same individual(s) and entity(ies) named as inventor(s) and applicant(s) on the present application. The Abstract and its presentation should be no impediment to a patent granting in any jurisdiction as to the subject matter claimed.

### STATEMENT AS TO FEDERALLY SPONSORED RESEARCH

This invention was made with government support under Grant No. AI084905 awarded by the National Institutes of Health. The government has certain rights in the invention.

### FIELD OF THE INVENTION

The invention relates to method of treating or inhibiting progression of hemoglobinopathy in a subject in need thereof comprising inhibiting interaction between LRF-BTB and CHD protein-LBD.

### BACKGROUND OF THE INVENTION

Genes encoding human β-type globin undergo a developmental switch from embryonic- to fetal- to adult-type. Mutations in the adult forms cause inherited hemoglobinopathies, or globin disorders, including sickle cell disease (SCD) and thalassemia, which some have suggested could be treated by re-induction of fetal-type hemoglobin (HbF). However, mechanisms that repress HbF in adults remain unclear. Applicants demonstrate that the LRF/ZBTB7A transcription factor occupies fetal γ-globin genes and maintains nucleosome density necessary for γ-globin gene silencing in adults. LRF confers its repressive activity through a NuRD repressor complex independent of the fetal globin repressor BCL11A. Applicants provide additional opportunities for therapeutic targeting in the treatment of hemoglobinopathies.

During human development, the site of erythropoiesis changes from the embryonic yolk sac to fetal liver and then, in newborns, to bone marrow, where it persists through adulthood. Coincidently, there is a “globin switch” from embryonic to fetal globin genes in utero, and then a second switch from fetal to adult globin expression soon after birth, a process studied extensively for over 60 years (G. Stamatoyannopoulos, Exp Hematol 33, 259-71 (2005)). The latter transition from fetal to adult hemoglobin is marked by a switch from a fetal tetramer consisting of two alpha and two gamma subunits (HbF:α2γ2) to an adult tetramer containing two α-like and two β-like globin subunits (HbA:α2β2).

Mutations in adult globin genes cause hemoglobinopathies such as thalassemia and sickle cell disease (SCD). These diseases are among the most common monogenic inherited human disorders and represent emerging public health challenges (S. Pleasants, Nature 515, S2-(2014)), for example, the number of children born with SCD is expected to exceed 14 million worldwide in the next 40 years (F. B. Piel, S. I. Hay, S. Gupta, D. J. Weatherall, T. N. Williams, PLoS Med 10, e1001484 (2013)).

Molecular genetics and clinical evidence indicates that elevated levels of fetal-type globin (HbF: α₂γ₂) in adults ameliorate SCD and β-thalassemia pathogenesis (Stamatoyannopoulos (2005), D. E. Bauer, S. C. Kamran, S. H. Orkin, Blood 120, 2945-53 (2012)). Thus, a promising approach is to pharmacologically inactivate a silencer(s) of fetal globin expression in order to re-activate HbF production in adult erythroid cells. Nuclear factors that regulate globin switching have been identified, but how they function cooperatively or independently in fetal globin repression is not fully understood.

### SUMMARY OF THE INVENTION

The invention provides a method of treating or inhibiting progression of hemoglobinopathy in a subject in need thereof comprising inhibiting interaction between LRF-BTB and CHD protein-LBD. In one aspect, the hemoglobinopathy comprises sickle cell anemia (SCD) or β-thalassemia. In a related aspect, the method comprises administering to the subject an effective amount of a small molecule, peptide, or antibody that inhibits the interaction in order to induce HbF expression. The invention generally provides methods to block LRF/CHD3 function by inhibiting the binding of these two proteins. Thus, the invention entails HbF production increase by inhibiting the silencer of HbF. In a further aspect, the method comprises an antibody which binds to LRF-BTB and/or CHD protein-LBD and/or CHD protein-domain and LRF-BTB when interacting. In an embodiment, the CHD protein comprises CHD3. In another embodiment, the CHD protein comprises CHD4.

In one aspect, the invention provides a method for identifying an agent as a potential inhibitor of LRF-BTB/CHD protein-LBD interaction for treating hemoglobinopathy comprising: (a) generating a series of N-terminal and C-terminal deletion mutations of the CHD protein-LBD consisting of an amino acid sequence having at least 95% identity with an amino acid sequence for CHD protein-LBD having SEQ ID NO. 2 or SEQ ID NO. 3, wherein the N-terminal and C-terminal deletion mutations are fragments; (b) measuring the binding of the fragments to the LRF-BTB domain; (c) comparing the interaction of the fragments to the LRF-BTB domain to a control to determine the relative strength of the binding between the fragments and the CHD protein CHD3/LRF-BTB domain or the CHD protein CHD4/LRF-BTB domain. In an aspect of the invention, the relative strength is measured by surface plasmon resonance.

In a related aspect, the invention provides a method for identifying an agent as a potential inhibitor of LRF-BTB/CHD protein CHD3-LBD interaction for treating hemoglobinopathy comprising: (a) co-introducing a first nucleic acid construct and a second nucleic acid construct into a cell or cell population, wherein the first nucleic acid construct comprises a nucleotide sequence which codes for a fusion protein which comprises an LRF-BTB domain linked to a reporter protein, wherein the LRF-BTB domain comprises an amino acid sequence having at least 95% identity with SEQ ID NO. 1, and the second nucleic acid construct comprises a nucleotide sequence which codes for a second fusion protein which comprises an CHD domain linked to a second reporter protein, wherein the CHD domain comprises an amino acid sequence for CHD3 or CHD4 having at least 95% identity with SEQ ID NO. 2 or 3; (b) allowing the cell or cell population to express the first fusion protein and the second fusion protein and contacting the cell or cell population with a potential inhibitor of LRF-BTB/CHD protein-LBD interaction; and, (c) detecting or quantifying an increase or decrease in protein complex formation, a change in subcellular localization, a concentration of signal or combination thereof, wherein a change in fluorescence indicates disruption of protein complex formation.

In an embodiment of the invention, the method comprises a reporter protein wherein the reporter protein is selected from the group consisting of a luciferase, a lactosidase, a green fluorescent protein, a yellow fluorescent protein, a cyan fluorescent protein and a red fluorescent protein. In a further embodiment, the reporter protein is humanized form of G. princeps luciferase. In an embodiment, the method comprises a cell or population of cells wherein the cell or cell population comprises HEK293 cells. In another embodiment, the method comprises identifying an agent as a potential inhibitor for treating hemoglobinapathy, wherein the hemoglobinopathy comprises sickle cell anemia, or β-thalassemia. In a related embodiment, the method comprises administering to the subject an effective amount of a small molecule, peptide, or antibody small molecule, peptide, or antibody. In a further embodiment, the method is performed as a medium- or high-throughput screening. In an embodiment, the method comprises identifying an agent as a potential inhibitor for treating hemoglobinapathy, wherein the CHD domain comprises an amino acid sequence for CHD3. In a further embodiment, the CHD domain comprises an amino acid sequence for CHD4.

In a related aspect, the invention provides an inhibitor of LRF-BTB/CHD protein CHD3-LBD interaction identified by any one of the methods provided herein. In an embodiment, the inhibitor comprises a small molecule, peptide, or antibody. The invention also provides an inhibitor of LRF-BTB/CHD protein CHD3-LBD interaction comprising a small molecule, peptide, or antibody.

In another aspect, the invention provides a method of determining a minimal amino acid sequence between an interaction of a CHD protein domain, wherein the CHD protein domain comprises an amino acid sequence having at least 95% identity with an amino acid sequence for CHD3 or CHD4 comprising SEQ ID NO. 2 or 3 and a LRF-BTB domain, wherein the LRF-BTB domain comprises an amino acid sequence having at least 95% identity with SEQ ID NO. 1, the method comprising: (a) generating a series of N-terminal and C-terminal deletion fragments of the LRF-BTB domain; (b) measuring the binding of the fragments to the CHD domain; and, (c) determining the relative strength of the binding between the fragments and the CHD domain. In an embodiment of the invention, the relative strength is measured by surface plasmon resonance.

In an aspect, the invention provides a method for identifying de-repressed γ-globin expression comprising: (a) introducing a vector that encodes and expresses a CHD protein/LRF-BTB domain fragment fused with a protein transduction domain into a cell or cell population, and (b) determining and/or measuring the γ-globin level of expression. In an embodiment of the invention, the method for identifying de-repressed γ-globin expression a vector wherein the vector comprises a lentiviral vector. In an embodiment, the method comprises a cell or cell population wherein the cell or cell population comprises HUDEP-2 cells. In another embodiment, the method wherein the protein transduction domain (PTD) comprises a PTD derived from a lentiviral TAT. In an embodiment, the method comprises de-repressed γ-globin expression, wherein the CHD domain comprises an amino acid sequence for CHD3. In a further embodiment, the CHD domain comprises an amino acid sequence for CHD4.

In certain embodiments, any of the aforementioned antibody/antibodies may be selected from, but not limited to, an IgG, IgA, or an antigen binding antibody fragment selected from an antibody single variable domain polypeptide, dAb, FAb, F(ab′)2, an scFv, an Fv, a V_{HH} domain (such as a Nanobody® or other camelized immunoglobulin domain) or a disulfide-bonded Fv. In certain embodiments, any of the above antibody types or fragments thereof may be prepared from one or more of a mammalian species selected from, but not limited to mouse, rat, rabbit, human. But of course, such antibodies should be humanized for use in humans. In certain embodiments, any of the above antibody types or fragments thereof may be provided as heteroconjugates, bispecific, single-chain, chimeric or humanized molecules having affinity for one or more specific CHD protein/LRF-BTB domains.

In certain embodiments, any of the hereinmentioned antibody/antibodies/small molecules/peptides binds to the domain, with a dissociation coefficient of 100 nM or less, 75 nM or less, 50 nM or less, 25 nM or less, such as 10 nM or less, 5 nM or less, 1 nM or less, or in embodiments 500 pM or less, 100 pM or less, 50 pM or less or 25 pM or less.

In certain embodiments, any of the hereinmentioned antibody/antibodies may be provided as polyclonal and/or monoclonal antibodies, including any mixture thereof.

Accordingly, the term “antibody”, unless the context so requires, includes polyclonal antisera and antibody cocktails as well as monoclonal antibodies. Antibodies may be monospecific, with narrow or broad specificity; or multispecific, such as bispecific, such that they possess two distinct epitope specificities in a single antibody molecule. Cocktails of antibodies may be targeted at two or more specific epitopes. Polyclonal antisera can be raised in a conventional manner against one or more antigens, and may include IgG, IgM and other antibody classes. In certain embodiments, antisera may be modified to comprise only IgG or only IgM. Antibody cocktails may be prepared by admixture of one or more monoclonal antibodies.

In one aspect, the antibody, small molecules, peptides of the invention are formulated for intravenous (iv) or intramuscular (im) administration. The small molecules are administered iv should extravasate from the circulation in order to enter the interstitial tissue space and bind to their cognate target.

The antibody, in one embodiment, is an antibody fragment such as a scFv, dAb or V_{HH} antibody. Small antibody fragments are extravasated much more readily into tissue, and for this reason can perform better than IgG or other larger antibodies. However, smaller fragments are also cleared faster from the circulation. A compromise must be struck between tissue accessibility and clearance. For example, see Wang et al., Clinical pharmacology & Therapeutics, 84:5, 2008, 548-558. Several antibody conjugates have been described which have extended half-life using a variety of strategies, for example through conjugation to albumin (such as human serum albumin). See Kontermann et al., BioDrugs April 2009, Volume 23, Issue 2, pp 93-109.

In one aspect the invention provides a viral vector system (e.g., AAV, retroviral, lentiviral) comprising nucleic acid molecule(s) encoding and expressing therapeutic, engineered protein/peptide compositions comprising e.g., antibodies, to target the protein-protein interface directly and/or via differential competition.

In one aspect the invention provides a method for viral delivery comprising administering to a subject at risk of developing sickle cell anemia or β-thalassemia or a subject suffering from sickle cell anemia or β-thalassemia, a viral vector system comprising nucleic acid molecule(s) encoding and expressing therapeutic, engineered protein/peptide compositions comprising e.g., antibodies, to target the protein-protein interface directly and/or via differential competition. In certain embodiments the viral vector system is an AAV system or a lentiviral system. Therapeutic compositions may also be delivered using a modified RNA system (Zangi et al., Nature Biotechnology 31, 898-907 (2013)).

In one embodiment, the cancer is that is any one or more of one or more carcinoma or sarcoma, including but not limited to cancers of the skin, pancreas, stomach, colon, thorax, liver, gallbladder, musculoskeletal system, breast, lung, ovary, uterus, endometrium, prostrate, colon, skin, mouth, salivary, esophagus, head and neck, plus other tumors of the gastrointestinal tract.

Accordingly, the inhibitors or agents of the invention which provide a method of treating a hemoglobinopathy can be delivered in combination with chemotherapy, which can achieve synergistic and/or curative results. Chemotherapeutic agents can be drugs or cytotoxic agents that inhibit or prevents the function of cells and/or causes destruction of cells. The drugs or cytotoxic agents may be targeted, or systemically administered. Examples of cytotoxic agents include radioactive isotopes, chemotherapeutic agents, and toxins such as small molecule toxins or enzymatically active toxins of bacterial, fungal, plant or animal origin, including synthetic analogues and derivatives thereof. The cytotoxic agent may be selected from the group consisting of an auristatin, a DNA minor groove binding agent, a DNA minor groove alkylating agent, an enediyne, a lexitropsin, a duocarmycin, a taxane, a puromycin, a dolastatin, a maytansinoid and a vinca alkaloid or a combination of two or more thereof. In one embodiment, the cancer is that is any one or more of one or more carcinoma or sarcoma, including but not limited to cancers of the skin, pancreas, stomach, colon, thorax, liver, gallbladder, musculoskeletal system, breast, lung, ovary, uterus, endometrium, prostrate, colon, skin, mouth, salivary, esophagus, head and neck, plus other tumors of the gastrointestinal tract

In one embodiment the drug is a chemotherapeutic agent selected from the group consisting of a topoisomerase inhibitor, an alkylating agent (eg. nitrogen mustards; ethylenimes; alkylsulfonates; triazenes; piperazines; and nitrosureas), an antimetabolite (eg mercaptopurine, thioguanine, 5-fluorouracil), an antibiotics (eg. anthracyclines, dactinomycin, bleomycin, adriamycin, mithramycin, dactinomycin) a mitotic disrupter (eg. plant alkaloids—such as vincristine and/or microtubule antagonists—such as paclitaxel), a DNA intercalating agent (eg carboplatin and/or cisplatin), a DNA synthesis inhibitor, a DNA-RNA transcription regulator, an enzyme inhibitor, a gene regulator, a hormone response modifier, a hypoxia-selective cytotoxin (eg. tirapazamine), an epidermal growth factor inhibitor, an anti-vascular agent (eg. xanthenone 5,6-dimethylxanthenone-4-acetic acid), a radiation-activated prodrug (eg. nitroarylmethyl quaternary (NMQ) salts) or a bioreductive drug or a combination of two or more thereof.

The chemotherapeutic agent may selected from the group consisting of Erlotinib (TARCEVA®), Bortezomib (VELCADE®), Fulvestrant (FASLODEX®), Sutent (SU11248), Letrozole (FEMARA®), Imatinib mesylate (GLEEVEC®), PTK787/ZK 222584, Oxaliplatin (Eloxatin®.), 5-FU (5-fluorouracil), Leucovorin, Rapamycin (Sirolimus, RAPAMUNE®.), Lapatinib (GSK572016), Lonafarnib (SCH 66336), Sorafenib (BAY43-9006), and Gefitinib (IRESSA®.), AG1478, AG1571 (SU 5271; Sugen) or a combination of two or more thereof.

The chemotherapeutic agent may be an alkylating agent—such as thiotepa, CYTOXAN® and/or cyclosphosphamide; an alkyl sulfonate—such as busulfan, improsulfan and/or piposulfan; an aziridine—such as benzodopa, carboquone, meturedopa and/or uredopa; ethylenimines and/or methylamelamines—such as altretamine, triethylenemelamine, triethylenephosphoramide, triethylenethiophosphoramide and/or trimethylomelamine; acetogenin—such as bullatacin and/or bullatacinone; camptothecin; bryostatin; callystatin; cryptophycins; dolastatin; duocarmycin; eleutherobin; pancratistatin; sarcodictyin; spongistatin; nitrogen mustards—such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide and/or uracil mustard; nitrosureas—such as carmustine, chlorozotocin, fotemustine, lomustine, nimustine, and/or ranimnustine; dynemicin; bisphosphonates—such as clodronate; an esperamicin; a neocarzinostatin chromophore; aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN®. doxorubicin—such as morpholino-doxorubicin, cyanomorpholino-doxorubicin, 2-pyrroline-doxorubicin and/or deoxydoxorubicin, epirubicin, esorubicin, idarubicin, marcellomycin, mitomycins—such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites—such as methotrexate and 5-fluorouracil (5-FU); folic acid analogues—such as denopterin, methotrexate, pteropterin, trimetrexate; purine analogues—such as fludarabine, 6-mercaptopurine, thiamiprine, thioguanine; pyrimidine analogues—such as ancitabine, azacitidine, 6-azauridine, carmofur, cytarabine, dideoxyuridine, doxifluridine, enocitabine, floxuridine; androgens—such as calusterone, dromostanolone propionate, epitiostanol, mepitiostane, testolactone; anti-adrenals—such as aminoglutethimide, mitotane, trilostane; folic acid replenisher—such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; ellliptinium acetate; an epothilone; etoglucid; gallium nitrate; hydroxyurea; lentinan; lonidainine; macrocyclic depsipeptides such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; razoxane; rhizoxin; sizofiran; spirogermanium; tenuazonic acid; triaziquone; 2,2′,2″-trichlorotriethylamine; trichothecenes—such as verracurin A, roridin A and/or anguidine; urethan; vindesine; dacarbazine; mannomustine; mitobronitol; mitolactol; pipobroman; gacytosine; arabinoside; cyclophosphamide; thiotepa; taxoids—such as TAXOL®. paclitaxel, abraxane, and/or TAXOTERE®, doxetaxel; chloranbucil; GEMZAR® gemcitabine; 6-thioguanine; mercaptopurine; methotrexate; platinum analogues—such as cisplatin and carboplatin; vinblastine; platinum; etoposide; ifosfamide; mitoxantrone; vincristine; NAVELBINE®, vinorelbine; novantrone; teniposide; edatrexate; daunomycin; aminopterin; xeloda; ibandronate; topoisomerase inhibitor RFS 2000; difluoromethylomithine (DMFO); retinoids—such as retinoic acid; capecitabine; and pharmaceutically acceptable salts, acids, derivatives or combinations of two or more of any of the above.

The drug may be a tubulin disruptor including but are not limited to: taxanes—such as paclitaxel and docetaxel, vinca alkaloids, discodermolide, epothilones A and B, desoxyepothilone, cryptophycins, curacin A, combretastatin A-4-phosphate, BMS 247550, BMS 184476, BMS 188791; LEP, RPR 109881A, EPO 906, TXD 258, ZD 6126, vinflunine, LU 103793, dolastatin 10, E7010, T138067 and T900607, colchicine, phenstatin, chalcones, indanocine, T138067, oncocidin, vincristine, vinblastine, vinorelbine, vinflunine, halichondrin B, isohomohalichondrin B, ER-86526, pironetin, spongistatin 1, spiket P, cryptophycin 1, LU103793 (cematodin or cemadotin), rhizoxin, sarcodictyin, eleutherobin, laulilamide, VP-16 and D-24851 and pharmaceutically acceptable salts, acids, derivatives or combinations of two or more of any of the above.

The drug may be a DNA intercalator including but are not limited to: acridines, actinomycins, anthracyclines, benzothiopyranoindazoles, pixantrone, crisnatol, brostallicin, CI-958, doxorubicin (adriamycin), actinomycin D, daunorubicin (daunomycin), bleomycin, idarubicin, mitoxantrone, cyclophosphamide, melphalan, mitomycin C, bizelesin, etoposide, mitoxantrone, SN-38, carboplatin, cis-platin, actinomycin D, amsacrine, DACA, pyrazoloacridine, irinotecan and topotecan and pharmaceutically acceptable salts, acids, derivatives or combinations of two or more of any of the above.

The drug may be an anti-hormonal agent that acts to regulate or inhibit hormone action on tumours—such as anti-estrogens and selective estrogen receptor modulators, including, but not limited to, tamoxifen, raloxifene, droloxifene, 4-hydroxytamoxifen, trioxifene, keoxifene, LY117018, onapristone, and/or fareston toremifene and pharmaceutically acceptable salts, acids, derivatives or combinations of two or more of any of the above. The drug may be an aromatase inhibitor that inhibits the enzyme aromatase, which regulates estrogen production in the adrenal glands—such as, for example, 4(5)-imidazoles, aminoglutethimide, megestrol acetate, AROMASIN®. exemestane, formestanie, fadrozole, RIVISOR®. vorozole, FEMARA®. letrozole, and ARIMIDEX® and/or anastrozole and pharmaceutically acceptable salts, acids, derivatives or combinations of two or more of any of the above.

The drug may be an anti-androgen—such as flutamide, nilutamide, bicalutamide, leuprolide, goserelin and/or troxacitabine and pharmaceutically acceptable salts, acids, derivatives or combinations of two or more of any of the above.

The drug may be a protein kinase inhibitor, a lipid kinase inhibitor or an anti-angiogenic agent.

The drug could also be a cytokine (e.g., an interleukin, a member of the TNF superfamily, or an interferon).

In a preferred embodiment, the drug is a maytansinoid, in particular DM1, or a tubulin disruptor.

It is an object or intention of the invention to not encompass within the scope of the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC) or any other patent office, tribunal or authority, such that Applicants reserve the right and hereby disclose a disclaimer of any product, process of making the product, or method of using the product that does not meet written description, enablement or sufficiency requirement(s) of the USPTO and/or EPO and/or other patent office, tribunal or authority.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

“Hemoglobinopathy” is a type of genetic defect resulting in abnormal struction of on of the globin chains of the hemoglobin molecule. Hemoglobinopathies are structural abnormalities in the globin proteins where as thalassemias are the result of underproduction of normal globin proteins, often through mutations in gregulatory genes.

As used herein, “cellular differentiation” or “differentiation” is the process by which a less specialized cell becomes a more specialized cell type.

“High-throughput screening” (HTS) refers to a process that uses a combination of modern robotics, data processing and control software, liquid handling devices, and/or sensitive detectors, to efficiently process a large amount of (e.g., thousands, hundreds of thousands, or millions of) samples in biochemical, genetic or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μl, 200 μl, 100 μl, 50 μl or less. Through this process, one can rapidly identify active compounds, small molecules, antibodies, proteins or polynucleotides which modulate a particular biomolecular/genetic pathway. The results of these experiments provide starting points for further drug design and for understanding the interaction or role of a particular biochemical process in biology. Thus “high-throughput screening” as used herein does not include handling large quantities of radioactive materials, slow and complicated operator-dependent screening steps, and/or prohibitively expensive reagent costs, etc.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, a “subject” means a human or animal (in the case of an animal, more typically a mammal, and can be, but is not limited to, a non-human animal or mammal). In one aspect, the subject is a human. A “subject” mammal can include, but is not limited to, a human or non-human mammal, such as a primate, bovine, equine, canine, ovine, feline, or rodent; and, it is understood that an adult human is typically about 70 kg, and a mouse is about 20 g, and that dosing from a mouse or other non-human mammal can be adjusted to a 70 kg human by a skilled person without undue experimentation.

The term “treating” is art-recognized and includes administration to the host or patient or subject of one or more of the subject compositions, e.g., to diminish, ameliorate, or stabilize the existing unwanted condition or side effects thereof. In aspects of the invention, treatment is for the patient or subject in need thereof.

By “agent” is meant a peptide, nucleic acid molecule, or small compound.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably more than a 30% change, a 35% change, a 40% change, and most preferably a 50% or greater change in expression levels. In a more preferred embodiment of the invention, the upregulation or increase in biomarker levels is at least greater than a 30% increase over baseline or normal population reference standards.

As used herein “detect” refers to identifying the presence, absence or amount of the analyte to be detected.

By “disease” is meant any condition or disorder that damages or interferes with the normal function of a cell, tissue, or organ. In one embodiment, the disease is hemoglobinopathy.

A “test agent” or “agent” refers to any molecule or compound, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, oligonucleotide, etc., to be tested for the capacity to directly or indirectly modulation tumor cell proliferation. The test agent can be in the form of a library of test compounds, such as a combinatorial or randomized library that provides a sufficient range of diversity. Test agents are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test agent (called a “lead agent”) with some desirable property or activity, e g, inhibiting activity, creating variants of the lead compound, and evaluating the property and activity of those variant compounds. Often, high throughput screening (HTS) methods are employed for such an analysis. Agents can be inhibitors, activators, or modulators of BaAdV nucleic acid and polypeptide sequences, and are used to refer to activating, inhibitory, or modulating molecules identified using in vitro and in vivo assays of the BaAdV nucleic acid and polypeptide sequences. Inhibitors are agents that, e.g., bind to, partially or totally block activity, decrease, prevent, delay activation, inactivate, desensitize, or down regulate the activity or expression of BaAdV, e.g., antagonists. Activators are agents that increase, open, activate, facilitate, enhance activation, sensitize, agonize, or up regulate BaAdV activity, e.g., agonists Inhibitors, activators, or modulators also include genetically modified versions of BaAdV, e.g., versions with altered activity, as well as naturally occurring and synthetic ligands, substrates, antagonists, agonists, antibodies, peptides, cyclic peptides, nucleic acids, antisense molecules, ribozymes, or small chemical molecules for example.

A “small organic molecule” or “small molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

As used herein, “antibody” refers to a polypeptide substantially encoded by an immunoglobulin gene or immunoglobulin genes, or antigen binding fragments thereof, which specifically binds and recognizes an analyte (antigen) such as adenovirus polypeptide or an antigenic fragment of thereof. Immunoglobulin genes include the kappa, lambda, alpha, gamma, delta, epsilon and mu constant region genes, as well as the myriad immunoglobulin variable region genes. Antibodies exist, for example as intact immunoglobulins and as a number of well characterized fragments produced by digestion with various peptidases. For instance, Fabs, Fvs, and single-chain Fvs (scFvs) that specifically bind to an adenovirus would be adenvirus-specific binding agents. A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (such as humanized murine antibodies), heteroconjugate antibodies such as bispecific antibodies). See also, Pierce Catalog and Handbook, 1994-1995 (Pierce Chemical Co., Rockford, Ill.); Kuby, J., Immunology, 3.sup.rd Ed., W.H. Freeman & Co., New York, 1997.

As used herein, the term “proteins” and “polypeptides” are used interchangeably herein to designate a series of amino acid residues connected to the other by peptide bonds between the alpha-amino and carboxy groups of adjacent residues. The terms “protein”, and “polypeptide”, which are used interchangeably herein, refer to a polymer of protein amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing

The term “prodrug” as used in this application refers to a precursor or derivative form of a compound of the invention that is capable of being enzymatically or hydrolytically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, ester-containing prodrugs, phosphate-containing prodrugs, thiophosphate-containing prodrugs, sulfate-containing prodrugs, peptide-containing prodrugs, D-amino acid-modified prodrugs, glycosylated prodrugs, .beta.-lactam-containing prodrugs, optionally substituted phenoxyacetamide-containing prodrugs, optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, compounds of the invention and chemotherapeutic agents such as described above.

A “metabolite” is a product produced through metabolism in the body of a specified compound or salt thereof. Metabolites of a compound may be identified using routine techniques known in the art and their activities determined using tests such as those described herein. Such products may result for example from the oxidation, hydroxylation, reduction, hydrolysis, amidation, deamidation, esterification, deesterification, enzymatic cleavage, and the like, of the administered compound. Accordingly, the invention includes metabolites of compounds of the invention, including compounds produced by a process comprising contacting a compound of this invention with a mammal for a period of time sufficient to yield a metabolic product thereof.

A “metabolite” is a product produced through metabolism in the body of a specified compound or salt thereof. Metabolites of a compound may be identified using routine techniques known in the art and their activities determined using tests such as those described herein. Such products may result for example from the oxidation, hydroxylation, reduction, hydrolysis, amidation, deamidation, esterification, deesterification, enzymatic cleavage, and the like, of the administered compound. Accordingly, the invention includes metabolites of compounds of the invention, including compounds produced by a process comprising contacting a compound of this invention with a mammal for a period of time sufficient to yield a metabolic product thereof.

A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

The term “polynucleotide” used herein refers to a polymer of deoxyribonucleotide or ribonucleotide that exists as a single-stranded or double-stranded form. The polynucleotide includes RNA genome sequences, DNA (gDNA and cDNA), and RNA sequences transcribed therefrom, and includes analogues of natural polynucleotides, unless specifically mentioned.

The polynucleotide also includes nucleotide sequences encoding the amino acid sequences of the heavy and light chain variable regions of an antibody disclosed herein, and nucleotide sequences complementary thereto. The complementary sequences include completely complementary sequences and substantially complementary sequences. For example, substantially complementary sequences are sequences that may be hybridized with nucleotide sequences encoding the amino acid sequences of the heavy or light chain variable regions of an antibody disclosed herein under stringent conditions known in the art. Stringent conditions mean, for example, hybridization to DNA in 6×SSC at about 45° C., followed by one or more washes in 0.2×SSC/0.1% SDS at about 50° C.-65° C.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker”, “biomarker” or “biological marker” is meant any clinical indicator, protein, metabolite, or polynucleotide having an alteration associated with a disease or disorder or a measurable indicator of some biological state or condition. Biomarkers are often measured and evaluated (e.g., whether their levels are increased or decreased or remain unchanged) to examine normal biological processes, pathogenic processes, or pharmacologic responses to a therapeutic intervention. In one embodiment, an alteration in body mass, lean body mass, metabolism, or a metabolite (e.g., clinical indicator) of disease state (e.g., hemoglobinopathy).

By “metabolic profile” is meant alterations in the level or activity of one or more amino acid, small molecule cellular metabolite, polypeptide or lipid metabolites.

As used herein, “obtaining” as in “obtaining an agent” includes synthesizing, purchasing, or otherwise acquiring the agent.

By “reduces” is meant a negative alteration of at least 10%, 25%, 50%, 70%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

It should be understood that proteins, including antibodies of the invention may associate with a specified region through various interactions to form ligand-receptor complexes. These interactions include but are not limited to electrostatic forces, such as hydrogen-bonding and Van der Waal forces, dipole-dipole interactions, hydrophobic interactions, pi-pi stacking, and so on. Other associations which describe more specific types of interactions include covalent bonds, electronic and conformational rearrangements, steric interactions, and so on. Thus, as used herein the term “associate” generally relates to any type of force which connects an antibody to a specified region. As used herein the term “interacts” generally relates to a more specific and stronger connection of an antibody to a specified region. As used herein the term “sterically blocks” is a specific type of association which describes an antibody interacting with a specific region and preventing other ligands from associating with that region through steric interactions. The terms “binds” or “specifically binds” as used throughout this application may be interpreted to relate to the terms “associates”, “interacts” or “sterically blocks” as required.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 illustrates illustrates the developmental switching of the β-type globin expression in human. Soon after the birth, fetal to adult globin switch occurs and only adult β-globin is expressed in adult. γ-globin is assembled with the two adult α-globin and forms the fetal hemoglobin tetramer, HbF, while adult β-globin forms adult hemoglobin HbA. HbF levels are less than 2% in adult.

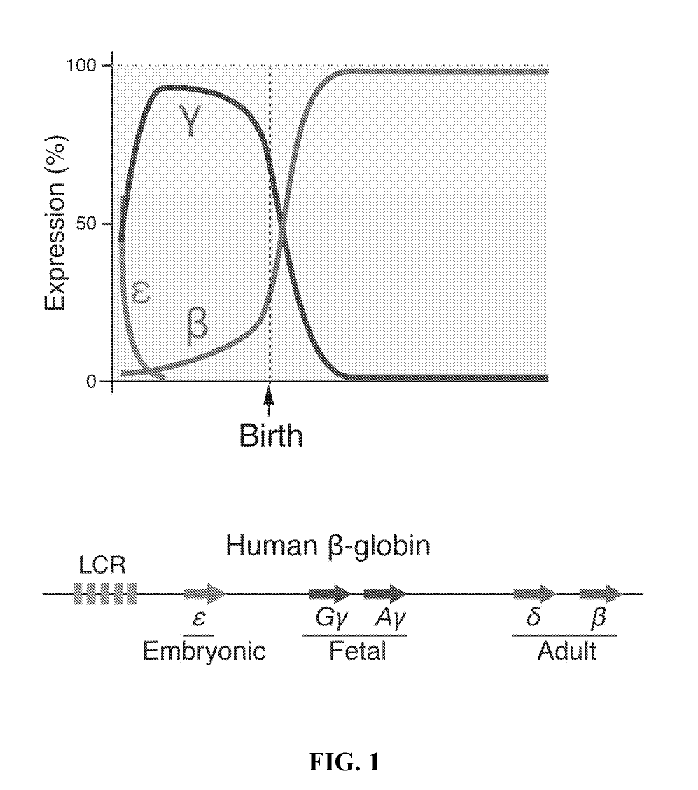

FIGS. 2A-2C illustrate induced Zbtb7a deletion reactivates embryonic/fetal globin expression in adult mice. (A) RNA-Seq analysis of control and LRF KO splenic erythroblasts. Mice were injected with pIpC and splenic erythroblasts harvested 2 months later. Each dot represents an individual gene, and differentially expressed genes are depicted based on FPM (Fragments Per Million mapped reads) values. (B) Isoelectric focusing of PB hemolysates and subsequent peptide mass fingerprinting with MALDI-TOFMS. Embryonic globins (Hbz and Hbbz) are evident in samples from LRF conditional KO mice. Globins shown in blue were detected at a much lower level. (C) Levels of human γ-globin (HBG) transcripts were monitored by q-PCR before and after LRF depletion. Error bars: standard deviation. We observed a low level of γ-globin expression prior to induction of LRF KO, likely due to leaky Cre activity (I. T. Chan, J. L. Kutok, I. R. Williams, S. Cohen, et al., Journal of Clinical Investigation 113, 528-38 (2004).

FIGS. 3A-3E illustrate ZBTB7A deletion reactivates γ-globin expression in human erythroblasts. (A) Time course analysis of LRF and BCL11A protein levels by Western blot. GAPDH: loading control. (B) Bar graphs show proportions of γ-globin to total β-globin transcripts measured by q-PCR on day 15. (C) Bar graphs show proportions of HbF relative to adult globin (HbA0) on day 15. Means of two independent samples per condition are shown. Error bars: standard deviation. (D) RNA-Seq analysis of control and LRF KO HUDEP-2 cells. Differentially-expressed genes are indicated based on FPM values. (E) Representative HPLC profiles of control and ZBTB7A^{Δ/Δ} HUDEP-2 clones. Control: HUDEP-2_Cas9.

FIG. 4 illustrates LRF occupies the γ-globin gene and maintains local chromatin compaction. LRF ChIP-Seq and ATAC-Seq signals at the β-globin cluster (HUDEP-2 cells). ChIP-Seq enrichment for GATA1, KLF1, and TAL1 (M. Y. Su, L. A. Steiner, H. Bogardus, T. Mishra, et al., J Biol Chem 288, 8433-44 (2013)) is also shown. Regions showing statistically significant ATAC-seq differences between LRF KO and WT HUDEP-2 cells are depicted in red. Because of high sequence similarity of HBG1 and HBG2, we analyzed LRF occupancy sites at the γ-globin locus using two different mapping methods: one mapping all mappable fragments (“LRF all”) and the other mapping only uniquely-mappable fragments (“LRF uniquely-mapped”). In the “LRF all” track, fragments mappable to either HBG1 or HBG2 were randomly distributed between both genes (marked by asterisks).

FIGS. 5A-5D illustrate LRF and BCL11A silence γ-globin expression through distinct mechanisms. (A) IP with anti-LRF antibody confirmed LRF/GATAD2B interaction in HSPC-derived erythroblasts. BCL11A was not detected in the LRF-containing NuRD complex. IP-sup: supernatant after IP (unbound fraction). (B) Reciprocal validation using anti-GATAD2B or anti-MTA2 antibody. (C) Representative HPLC profiles of control (HUDEP-2_Cas9), ZBTB7A KO, BCL11A KO, and ZBTB7A/BCL11A DKO (clone #5 and #18) HUDEP-2 cells. (D) Bar graphs show proportions of HbF relative to adult globin (HbA0). Means of two independent samples per clone are shown. Error bars: standard deviation.

FIGS. 6A-6B illustrate efficient Zbtb7a deletion in splenic erythroblasts of Zbtb7a^{F/F} Mx1-Cre+ mice. (A) LRF protein was not detected by Western blot in splenic erythroblasts of Zbtb7a^{F/F} Mx1-Cre+ mice. Splenic erythroblasts were obtained one month after pIpC injection. Hdac1: loading control. (B) RNA-Seq read profiles at the Zbtb7a locus. No signals from exon2 were detected in LRF KO samples, verifying efficient Zbtb7a deletion. Two loxp sites were introduced to flank Zbtb7a exon2 in our Zbtb7a conditional knockout model (T. Maeda, T. Merghoub, R. M. Hobbs, L. Dong, et al., Science 316, 860-6 (2007)).

FIGS. 7A-7D illustrate RNA-Seq analysis of LRF-deficient mouse splenic erythroblasts. (A) Bar graphs show levels of embryonic- and adult-globin transcripts in control (F/F) and LRF KO (F/F Cre+) basophilic- and polychromatophilic (CD71⁺TER119⁺CD44^{med}FSC^{med})-erythroblasts. y-axis represents mean FKPM values obtained from RNA-Seq experiments (two independent samples per genotype). Values in parentheses indicate mean FKPM values of less abundant transcripts. P values: unadjusted P values determined using the DESeq Bioconductor package (S. Anders, W. Huber, Genome Biol 11, R106 (2010)). (B) Relative Hbb-bh1 mRNA levels in splenic erythroblasts as determined by q-PCR. Hbb-bh1 transcript levels in LRF-deficient erythroblasts were 1714× higher than those seen in controls. (C) LRF inactivation in β-YAC mice. (D) Levels of human embryonic β-globin (HBE1) transcripts were monitored by q-PCR as shown in FIG. 2C.

FIGS. 8A-8E illustrate LRF inactivation induces γ-globin production in HSPC-derived erythroid cells. (A) Human CD34+ cells were induced into an erythroid lineage with EDM-based medium (see, e.g., Methods). EDM-I: EDM supplemented with hydrocortisone, SCF and IL3; EDM-II: EDM with SCF; and EDM-III: EDM with no supplement. Lentivirus (pLKO vector) infection was performed on day 7 and puromycin selection started on day 9. (B) Protein samples were obtained from day 15 erythroblasts. LRF knockdown efficiency was assessed by Western blot using the anti-LRF antibody. GAPDH: loading control. (C) Relative copy number of indicated β-globin transcripts was determined by q-PCR. A plasmid containing the corresponding PCR amplicon was generated for each gene and used to calculate copy number. Copy numbers are reported relative to corresponding RPS18 levels in each sample. (D) Representative γ-globin FACS profiles of untreated, scrambled shRNA-treated, and LRF shRNA-treated human erythroblasts. Each sample was first incubated with either non-specific mouse IgG1κ (control) or anti-HBG1/2 antibody, followed by staining with fluorochrome-conjugated anti-mouse IgG1 antibody. γ-globin positivity was determined based on background signal levels (IgG control). (E) Representative HPLC profiles on day 15. % HbF was defined as a proportion of HbF relative to HbA0.

FIGS. 9A-9B illustrate effects of LRF inactivation on HSPC-derived erythroid differentiation. (A) ShRNA-mediated LRF knockdown promotes a delay in erythroid differentiation. Representative FACS profiles of Day 10 and 14 erythroblasts are shown. Fewer CD235+CD71+ cells were evident on day 10 in LRF knockdown relative to control cells. (B) Representative images of Wright-Giemsa staining of cytospins prepared on Day 14.

FIGS. 10A-10D illustrate LRF inactivation in HUDEP-2 cells. (A) HUDEP-2 cells were maintained in SFEM medium in the presence of doxycycline (Dox) as described (R. Kurita, N. Suda, K. Sudo, K. Miharada, el al., PLoS One 8, e59890 (2013)). To induce hemoglobin production, cells were cultured with EDM-II with Dox for 5 days and then cultured without Dox for two more days prior to analysis. (B) LRF KO in HUDEP-2 cells was validated by Western blot. BCL11A levels were also examined using BCL11A knockdown HUDEP-2 cells as a control. Tubulin: loading control. (C) Representative images of Wright-Giemsa staining of cytospins prepared on indicated days. (D) Immunophenotyping of control and LRF KO (ZBTB7A^{Δ/Δ}) HUDEP-2 cells. FACS profiles of indicated surface markers were indistinguishable between genotypes.

FIG. 11 illustrate HUDEP-2 cells exhibit gene expression patterns similar to those seen in human basophilic erythroblasts. Principal component analysis was performed using the RNA-Seq data (log₁₀ read counts) of HUDEP-2 cells (this study) and HSPC-derived erythroid cells (57, 58). First (PC1) and third (PC3) components are shown, since the second (PC2) component captured separation of the HUDEP cells relative to all other datasets.

FIGS. 12A-12F illustrate LRF inactivation induces γ-globin expression in HUDEP-2 cells. (A) RNA-Seq analysis of control and LRF KO HUDEP-2 cells before (SFEM, Dox+) and after (EDM day 5, Dox+) differentiation. Bar graphs show mRNA levels (mean FPKM values) of α- and β-globins in control (ZBTB7A^{+/+}) and LRF KO (ZBTB7A^{Δ/Δ}) HUDEP-2 cells. Values in parentheses indicate mean FKPM values of less abundant transcripts. P values: unadjusted P values determined using the DESeq Bioconductor package (Anders 2015). (B) Bar graphs show proportions of γ-globin mRNA to total β-globin mRNA in control and LRF KO HUDEP-2 cells. Control: HUDEP-2_Cas9. (C) γ-globin protein levels were determined by Western blot using protein lysates from control, ZBTB7A^{Δ/Δ} and BCL11A^{Δ/Δ} HUDEP-2 cells. HBG protein was detected only in ZBTB7A^{Δ/Δ} or BCL11A^{Δ/Δ} cells. GAPDH: loading control. (D) Representative FACS profiles of γ-globin protein expression. (E) LRF protein was not detected in ZBTB7A^{Δ/Δ} clones. HSP90: loading control. Asterisk denotes band corresponding to a residual LRF signal from a primary blot. (F) Bar graphs show transcript levels of known γ-globin repressors in control and ZBTB7A^{Δ/Δ} HUDEP-2 cells. Mean FPKM values of two independent samples per genotype (before differentiation) are shown.

FIGS. 13A-13C illustrate LRF ChIP-Seq in human erythroid cells. (A) LRF-ChIP-Seq was performed using HSPC-derived erythroblasts (Day 8) in duplicate and HUDEP-2 cells in triplicates. Distribution of sample correlation coefficients among replicates was examined. Pearson's correlation coefficient values are shown. (B) Analysis revealed 5684 and 10385 LRF binding sites in human erythroblasts and HUDEP-2 cells, respectively. Venn diagram chart shows number of target genes shared between both cell types. (C) Heat map showing correlation between LRF (this study) and BCL11A and SOX6 (Xu 2010) binding sites. LRF and BCL11A occupancy sites are mutually exclusive.

FIG. 14 illustrates LRF binding motifs identified in HSPC-derived erythroid cells. LRF binding motifs were identified using motif discovery programs (Tomtom (S. Gupta, J. A. Stamatoyannopoulos, T. L. Bailey, W. S. Noble, Genome Biol 8, R24 (2007)), MEME (T. L. Bailey, C. Elkan, Proc Int Conf Intell Syst Mol Biol 2, 28-36 (1994)), DREAME (T. L. Bailey, Bioinformatics 27, 1653-9 (2011)), CentriMo (T. L. Bailey, P. Machanick, Nucleic Acids Res 40, e128 (2012)), TRANSFAC (V. Matys, O. V. Kel-Margoulis, E. Fricke, I. Liebich, et al., Nucleic Acids Res 34, D108-10 (2006)) and JASPAR (A. Mathelier, X. Zhao, A. W. Zhang, F. Parcy, et al., Nucleic Acids Res 42, D142-7 (2014))). Motifs identified with high statistical significance (E-value<5×10̂−3 are listed. Similar known motifs are also shown. Motif distribution curve (Bailey 2012) was generated based on the probability of the best match to a given motif occurring at a given position in input sequences. Grey vertical line indicates center of input sequence.

FIG. 15 illustrates LRF binding motifs identified in HUDEP-2 cells. Motif discovery was performed using HUDEP-2 LRF-ChIP-Seq data as described.

FIG. 16A-16B illustrates LRF-interacting proteins identified by Y2H. (A) A Y2H screen identified components of the NuRD complex and chromatin remodelers as direct LRF binding proteins. LRF-BTB dimer structure based on a 2NN2 PDB file is shown. (Move panel A to the SOM) (B) Proteins listed were identified as direct LRF binding partners with high confidence. Protein domain architectures were obtained from SMRT (http://smart.embl.de/). The LRF binding region in each protein is depicted with a purple oval.

FIGS. 17A-17F illustrate interaction between LRF and NuRD components. (A) Bar graphs show abundance of indicated transcripts in mouse erythroblasts (left) and HUDEP-2 cells (right). FKPM values were obtained from RNA-Seq experiments. (B) Validation of Y2H results by IP. Exogenously-expressed Flag-tagged target proteins interact with endogenous LRF in 293T cells. Flag-tagged target proteins (CHD8, CHD3, BAZ1A or GATAD2B) were expressed in 293T cells, lysates were prepared 48 h later and IP was performed using standard methodology with anti-Flag antibody, followed by Western blot with anti-LRF antibody. Protein samples from empty vector-transfected cells served as negative controls. (C) LRF/GATAD2B interaction was validated in mouse erythroleukemia (MEL) cells. Applicants generated a series of expression vectors encoding N- or C-terminally-tagged mouse LRF (mLRF) and performed IP in MEL cells. Applicants could pull-down NuRD components and endogenous LRF protein only when Applicants expressed the LRF tagged at the far C-terminus. Anti-HA antibody could pull down both endogenous and exogenous LRF plus NuRD components (far right lane), while antibody against Flag, which was placed 15 aa upstream of the HA tag, pulled down only exogenous LRF (2nd lane from the left). Although Applicants tested 5 different (2 original and 3 commercially-available) anti-LRF antibodies for IP in mouse cells, none could pull down endogenous mouse LRF protein. Native LRF in the large NuRD complex may be inaccessible to anti-LRF antibodies in mouse cells (a model shown on right). Applicants note that the BCL11A image is from a different membrane. (D) Knockout of LRF or BCL11A was confirmed by Western blot using anti-LRF or -BCL11A antibodies. GAPDH: loading control. (E) Relative copy number of indicated β-globin transcripts was determined by q-PCR, as described in FIG. 8C. (F) Bar graphs show proportions of γ-globin mRNA to total β-globin mRNA in control and LRF KO HUDEP-2 cells. Control: HUDEP-2_Cas9.

FIG. 18 illustrates a Proposed model for γ-globin silencing mediated by the LRF-NuRD complex.

FIGS. 19A-19B illustrate Gene Set Enrichment Analysis (GSEA) of gene expression differences following ZBTB7A knockout. (A) GSEA was performed based on log₂ fold-differences in expression magnitude between control and Zbtb7a KO splenic erythroblasts (FIG. 2A). Listed are gene Ontology (GO categories showing statistically significant association after correction for multiple hypothesis testing (Q-value). A positive Edge value indicates upregulated gene expression in LRF knockout cells. (B) Shown are significantly associated GO categories for expression differences between control and ZBTB7A^{Δ/Δ} HUDEP-2 cells (EDM-II Dox+ Day 5).

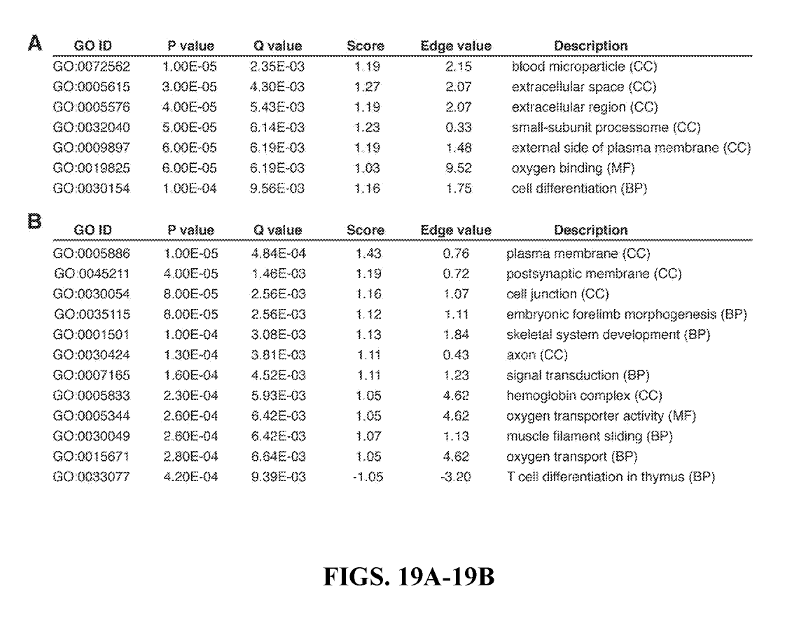

### DETAILED DESCRIPTION OF THE INVENTION

Hemoglobinopathies, such as sickle cell disease (SCD) and thalassemia, are among the greatest public health concerns in the world. Although new therapeutic modalities, such as gene therapy, are currently being tested, a pharmacologic approach is absolutely needed for general patient populations. Molecular genetics and clinical evidence indicates that elevated levels of fetal-type globin (HbF: α2γ2) in adults ameliorate SCD and β-thalassemia pathogenesis. Thus, a promising approach is to pharmacologically inactivate a silencer(s) of fetal globin expression in order to re-activate HbF production in adult erythroid cells.

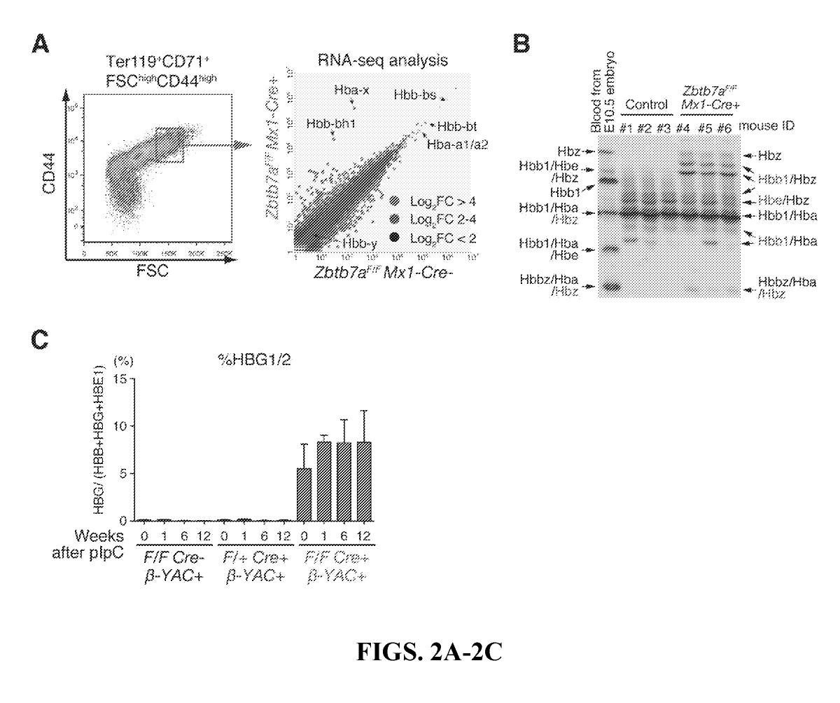

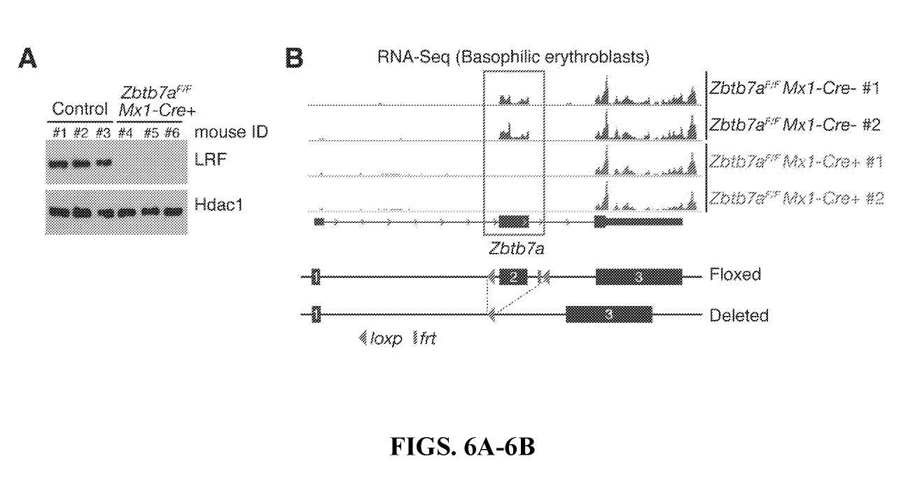

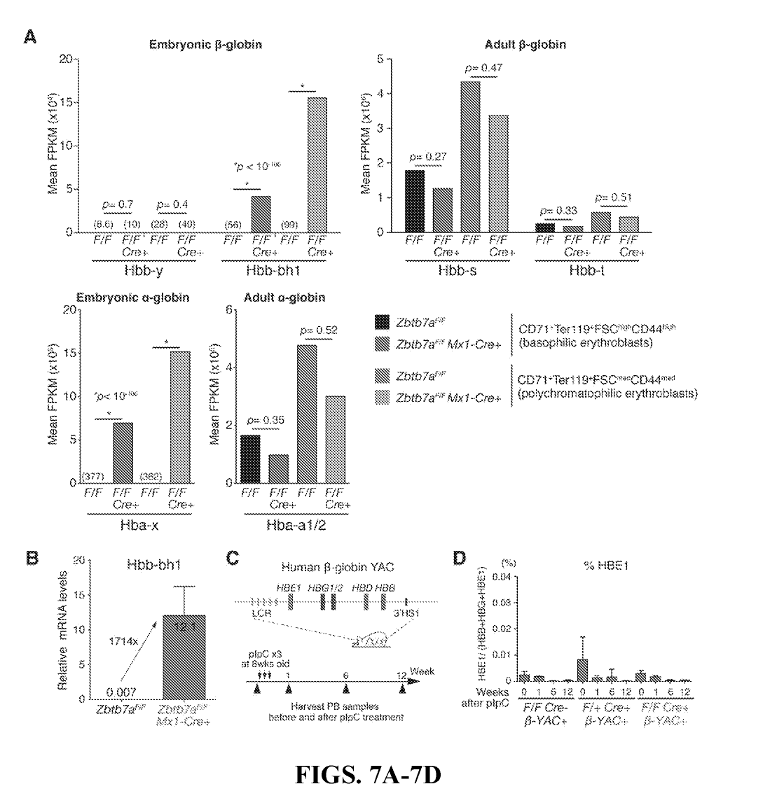

Leukemia/lymphoma related factor (LRF), encoded by the ZBTB7A gene, is a ZBTB transcription factor that binds DNA through C-terminal C2H2-type zinc fingers and presumably recruits a transcriptional repressor complex through its N-terminal BTB domain (S. U. Lee, T. Maeda, Immunol Rev 247, 107-19 (2012)). To assess the effects of LRF loss on the erythroid transcriptome, Applicants inactivated the Zbtb7a gene in erythroid cells of adult mice (S. U. Lee, M. Maeda, Y. Ishikawa, S. M. Li, et al., Blood 121, 918-29 (2013)). Applicants then performed RNA-Seq analysis using splenic erythroblasts from control and LRF conditional knockout (Zbtb7a^{F/F} Mx1-Cre+) mice (FIG. 2A). Efficient Zbtb7a deletion was confirmed by Western blot and RNA-Seq reads (FIGS. 6A and 6B) (see, e.g., Materials and Methods in Examples). Wild type (WT) mice express two embryonic β-like globin genes: Hbb-bh1 and Hbb-y (P. D. Kingsley, J. Malik, K. A. Fantauzzo, J. Palis, Blood 104, 19-25 (2004),). Although both genes are expressed at early embryonic stages, Hbb-bh1 is the orthologue of human γ-globin (K. Chada, J. Magram, F. Costantini, Nature 319, 685-9 (1986), P. D. Kingsley, J. Malik, R. L. Emerson, T. P. Bushnell, et al., Blood 107, 1665-72 (2006)). LRF-deficient adult erythroblasts showed significant induction of Hbb-bh1, but not Hbb-y, with a moderate reduction in adult globin levels (FIG. 7A). These results were validated by q-PCR (FIG. 7B). Isoelectric focusing of peripheral blood (PB) hemolysates revealed unique bands corresponding to embryonic globin proteins in PB from LRF conditional knockout (KO) mice (FIG. 2B).

Applicants next asked whether LRF loss would reactivate human fetal globin expression in vivo by employing a humanized mouse model. To do so, Applicants established LRF conditional KO mice harboring the human β-globin gene cluster as a yeast artificial chromosome transgene (β-YAC) (K. R. Peterson, C. H. Clegg, C. Huxley, B. M. Josephson, et al., Proceedings of the National Academy of Sciences 90, 7593-7 (1993)) (FIG. 7C). Human γ-globin transcripts, but not those of embryonic β-globin (HBE1), were significantly induced in LRF-deficient erythroblasts and comprised 6-12% of total human β-like globins in PB (FIGS. 2C and 7D). The magnitude of globin induction in LRF/βYAC mice approximated that seen in BCL11A/βYAC mice (J. Xu, C. Peng, V. G. Sankaran, Z. Shao, et al. Science 334, 993-6 (2011)).

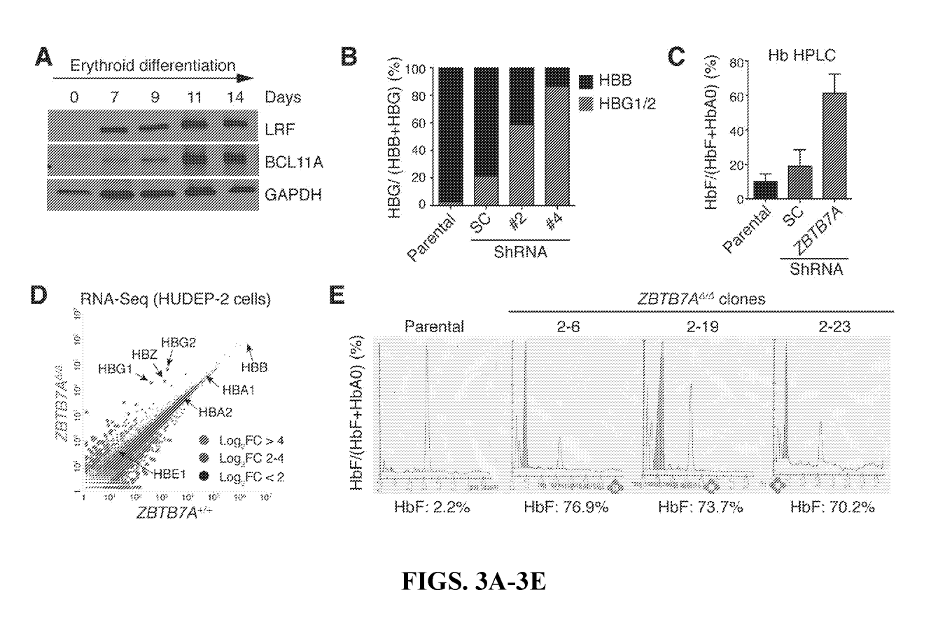

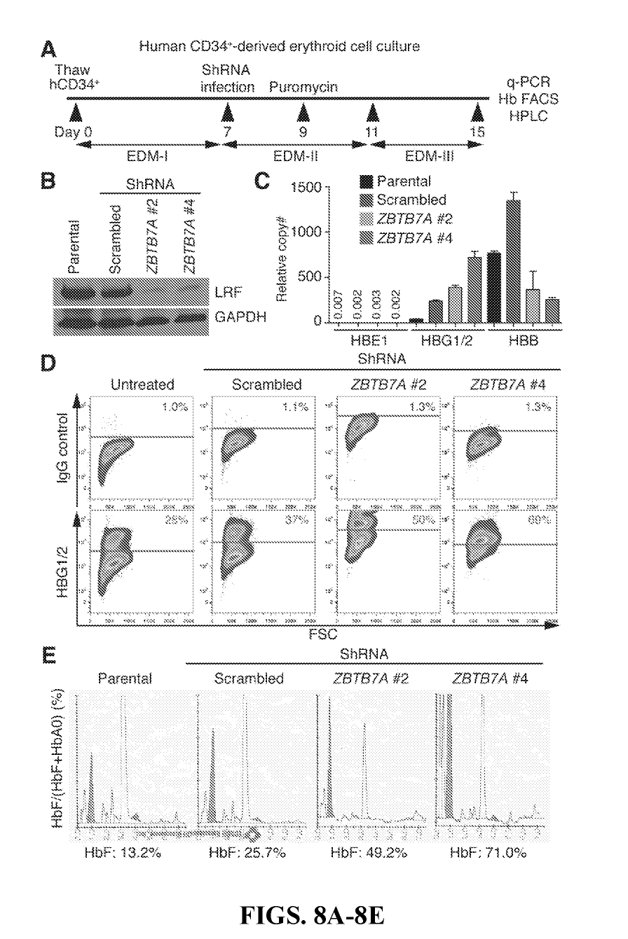

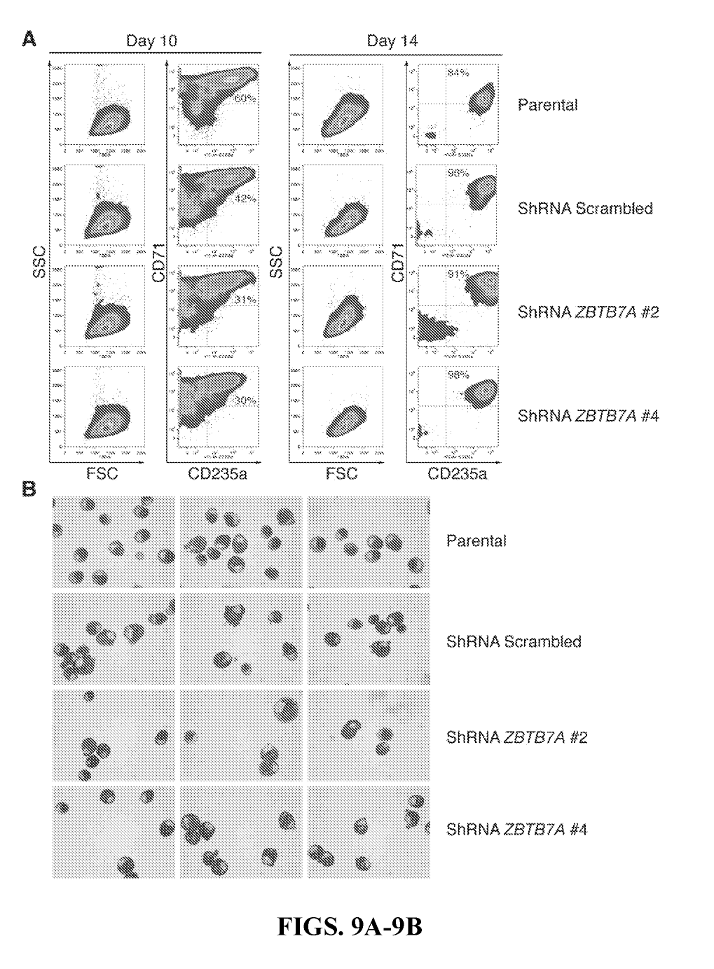

Applicants next determined whether LRF loss could induce HbF in human erythroid cells. To this end, Applicants employed human CD34⁺ hematopoietic stem and progenitor (HSPC)-derived primary erythroblasts and determined γ-globin expression levels upon shRNA-mediated LRF knockdown (KD) (FIG. 8A). LRF expression was markedly induced upon erythroid differentiation over a two-week period (FIG. 3A). LRF KD significantly increased the percentage of γ-globin mRNA (FIGS. 3B, 8B and 8C) and protein expression (FIG. 8D) relative to adult globin. HbF levels in LRF KD cells were greater than those seen in parental or scrambled-shRNA transduced cells (FIGS. 3C and 8E). Since LRF conditional KO mice exhibit a mild macrocytic anemia due to inefficient erythroid terminal differentiation (T. Maeda, K. Ito, T. Merghoub, L. Poliseno, et al., Dev Cell 17, 527-40 (2009)). Applicants assessed the effects of LRF deficiency on human erythroid differentiation. Applicants observed a delay in differentiation upon LRF KD compared to controls (FIG. 9A, 9A and Supplementary Text).

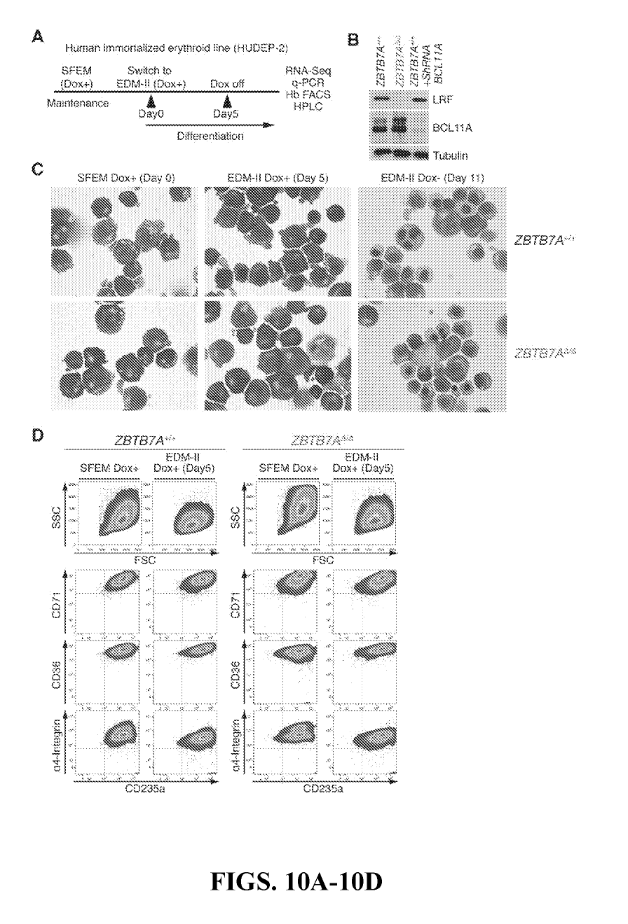

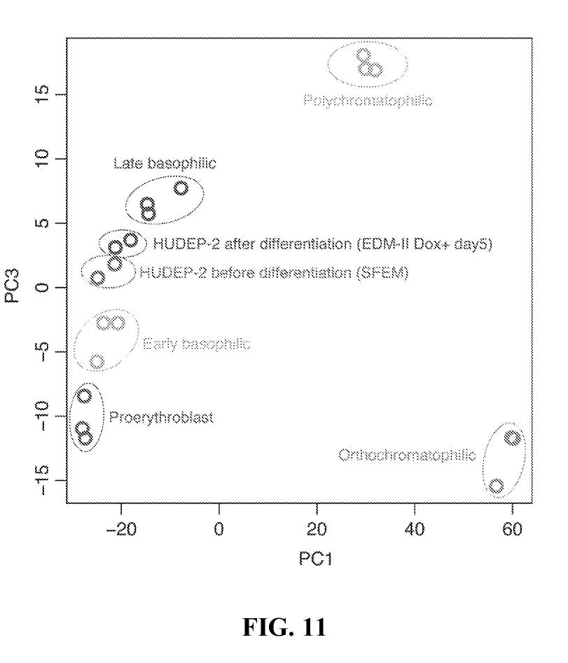

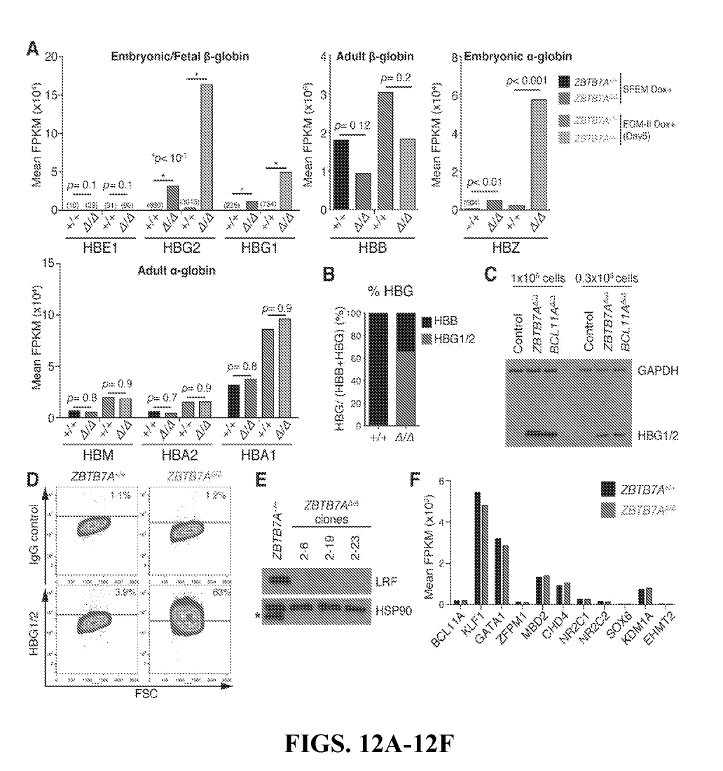

HSPC-derived erythroid cells tend to have relatively high basal levels of HbF (FIG. 3C). Moreover, it is difficult to exclude that the effects of LRF KD may be the result of a subpopulation of cells expressing aberrantly high HbF levels. To circumvent these problems, Applicants employed a human immortalized erythroid line (HUDEP-2), which undergoes terminal differentiation upon doxycycline removal (FIG. 10A) (Kurita 2013). This line possesses an advantage over lines currently used for globin switching studies because it expresses predominantly adult hemoglobin (HbA: α₂β₂), with very low background HbF expression (R. Kurita, N. Suda, K. Sudo, K. Miharada, et al., PLoS One 8, e59890 (2013)). Using CRISPR/cas9 gene modification, Applicants knocked out ZBTB7A in HUDEP-2 cells (FIG. 10B). Applicants did not observe a significant difference in erythroid differentiation between control and ZBTB7A KO HUDEP-2 cells, as evidenced by morphologic and FACS analyses (FIGS. 10C and 10D). To evaluate genome-wide gene expression changes promoted by ZBTB7A deletion, Applicants performed RNA-Seq analysis, WT HUDEP-2 cells exhibit gene expression patterns similar to those of HSPC-derived basophilic erythroblasts (FIG. 11). As expected, γ-globin transcripts, but not those of embryonic ε-globin, were markedly induced in ZBTB7A KO HUDEP-2 cells (FIGS. 3D and 12A). Levels of adult β-globin transcripts in ZBTB7A KO cells were approximately half those seen in control cells (FIG. 12A). γ-globin transcripts comprised more than 60% of total β-like globins (FIG. 12B). Induction of γ-globin was also validated at the protein level (FIGS. 12C and 12D). Applicants then established three independent ZBTB7A KO HUDEP-2 clones (FIG. 12E) and determined HbF levels in each by HPLC. All three clones exhibited HbF levels greater than 60%, whereas that of parental cells was less than 3% (FIG. 3E). Notably, the HbF reactivation occurred without changes in levels of transcripts encoding known HbF repressors, including BCL11A (FIG. 12F). BCL11A protein levels were also unchanged in ZBTB7A KO cells (FIG. 10B).

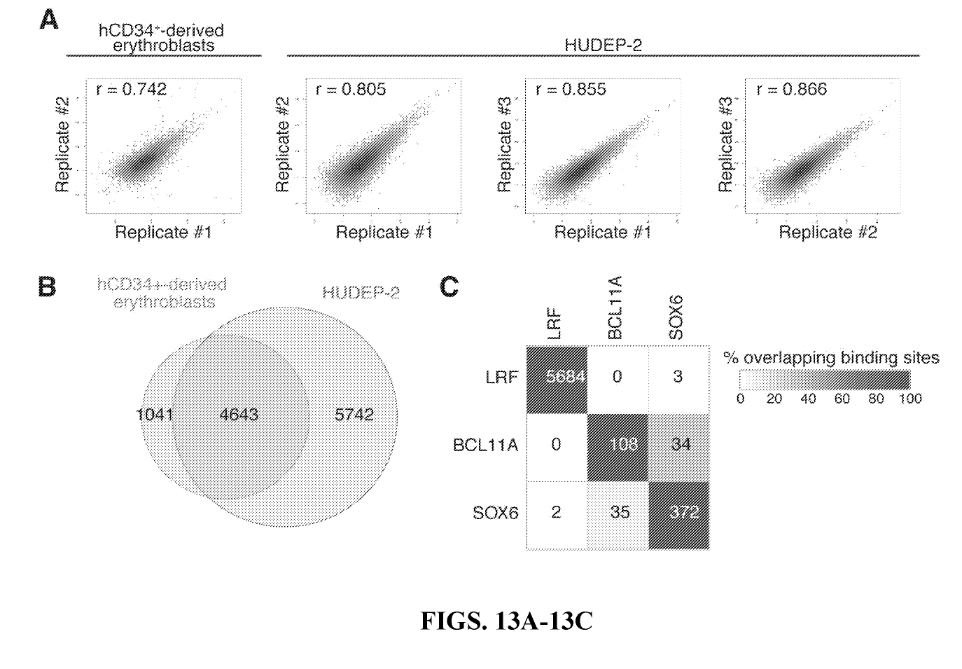

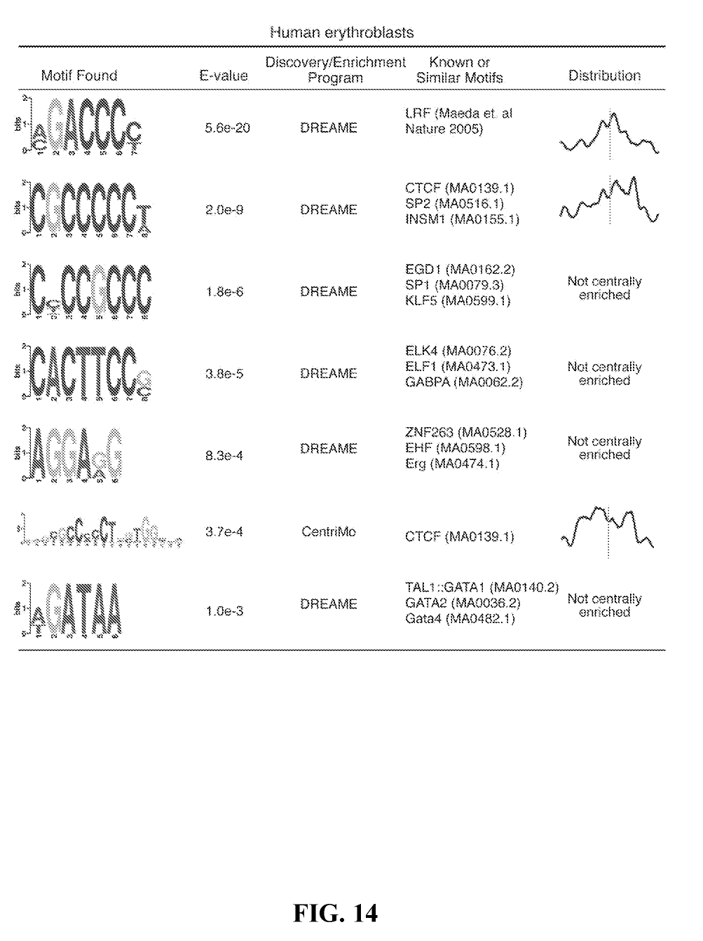

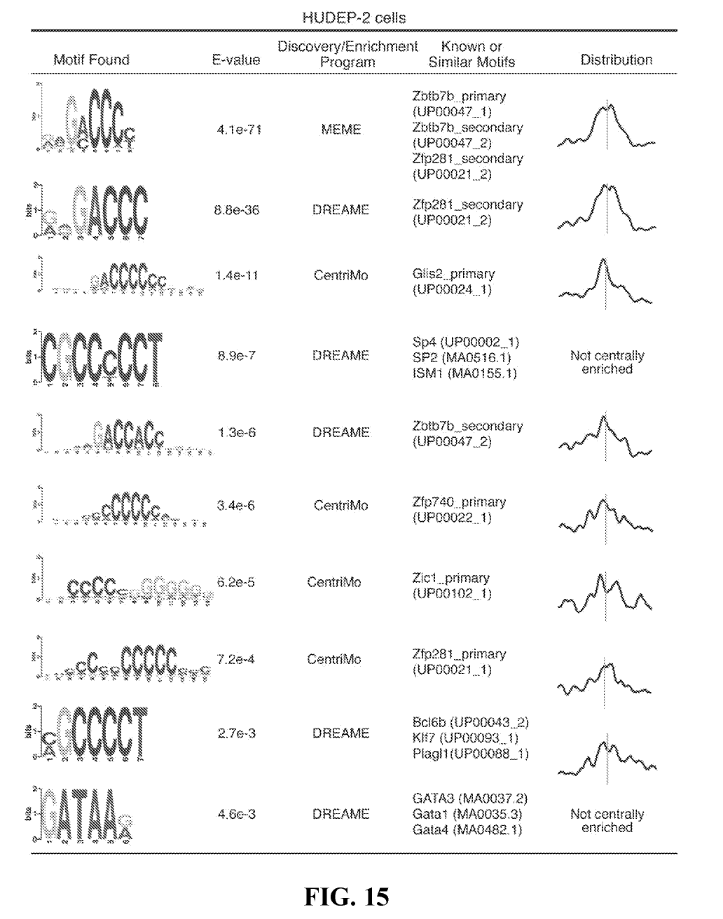

To determine LRF occupancy sites genome-wide, Applicants performed chromatin-immunoprecipitation and sequencing (ChIP-Seq) with an anti-LRF antibody using HSPC-derived proerythroblasts and HUDEP-2 cells. These experiments exhibited strong correlations and concordance among the replicates (FIG. 13A). Applicants identified 5,684 and 10,385 LRF binding sites in HSPC-derived proerythroblasts and HUDEP-2 cells, respectively (FIG. 13B). The most enriched motif identified in either cell type was consistent with that previously identified in vitro using CAST (cyclic amplification and selection of targets) analysis (T. Maeda, R. M. Hobbs, T. Merghoub, I. Guernah, et al., Nature 433, 278-85 (2005)), confirming antibody specificity (FIGS. 14 and 15). Genes differentially expressed in control and ZBTB7A KO cells were significantly enriched for LRF binding sites (Fisher's exact test p<1.6×10⁻⁵ and p<8.3×10⁻¹³ for undifferentiated and differentiated conditions, respectively). It is also notable that LRF occupancy sites differ from those of known γ-globin repressors SOX6 and BCL11A (J. Xu, V. G. Sankaran, M. Ni, T. F. Menne, et al., Genes Dev 24, 783-98 (2010)) (FIG. 13C).

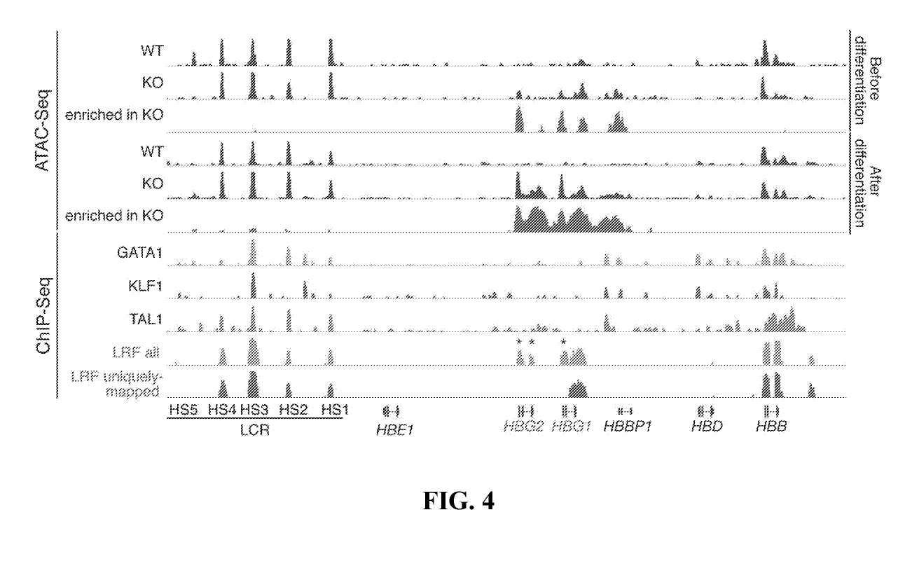

In support of a direct role of LRF at the β-globin cluster, Applicants observed several significant LRF-ChIP binding signals at adult and fetal globin genes and at upstream hypersensitivity (HS) sites within the locus control region (LCR) (“LRF uniquely-mapped” track in FIG. 4). LRF may also bind to HBG2; however, the high degree of sequence homology between HBG1 and HBG2 did not allow us to assess LRF binding at HBG2 independently of HBG1 (FIG. 4). To assess the local chromatin accessibility at the β-globin cluster in the presence or absence of LRF, Applicants performed ATAC-Seq (for assay for transposase-accessible chromatin with high-throughput sequencing (J. D. Buenrostro, P. G. Giresi, L. C. Zaba, H. Y. Chang, W. J. Greenleaf, Nat Methods (2013)). In control HUDEP-2 cells, the HBB gene and LCR HS sites, but not the γ-globin gene, exhibit ATAC-Seq nucleosome-free signals (FIG. 4). In contrast, strong chromatin accessibility was evident at the γ-globin genes in ZBTB7A^{Δ/Δ} cells before differentiation, and the signal was amplified upon differentiation (FIG. 4). Strikingly, differential enrichment of ATAC-signals in ZBTB7A^{Δ/Δ} cells was evident only at the γ-globin genes (FIG. 4) but not at the HBB gene or HS sites, indicating that chromatin in the latter is accessible, regardless of ZBTB7A genotype. Thus, while LRF binds to the HBB gene and HS sites as well as to the HBG1 gene, LRF depletion specifically opens chromatin at the γ-globin genes.

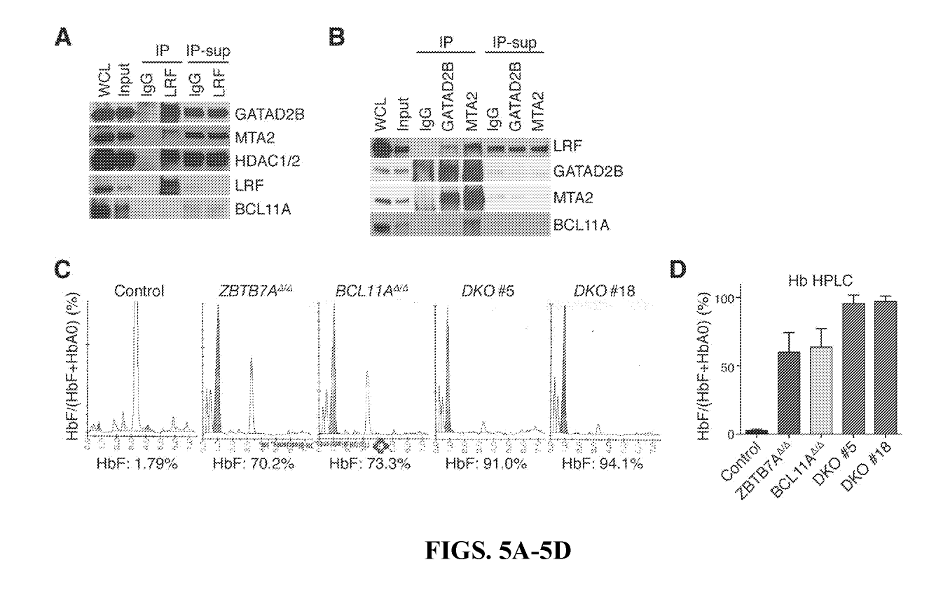

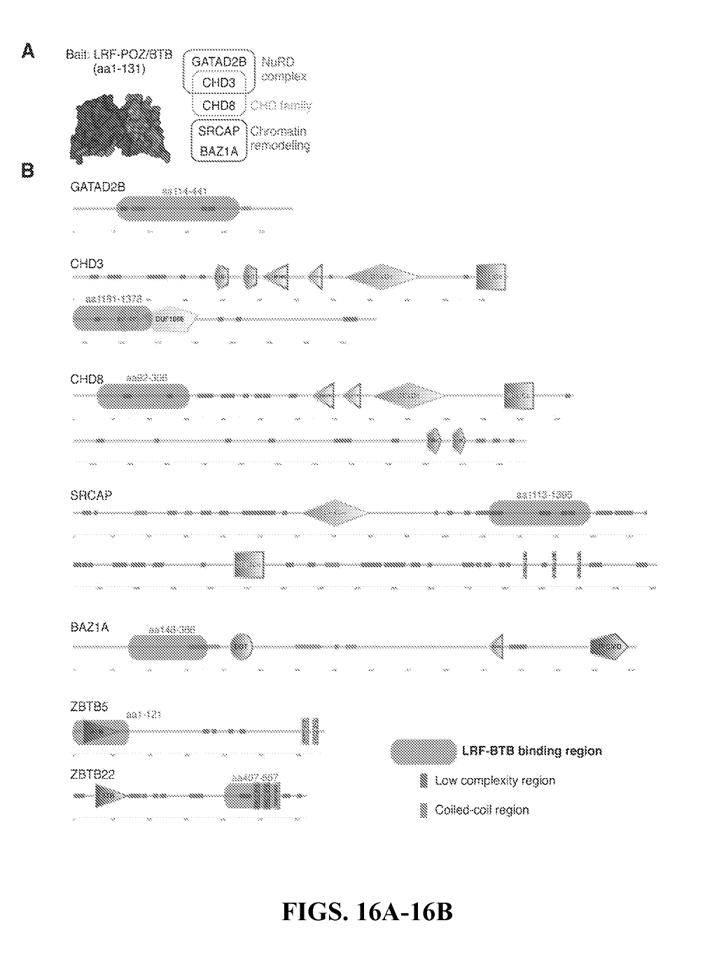

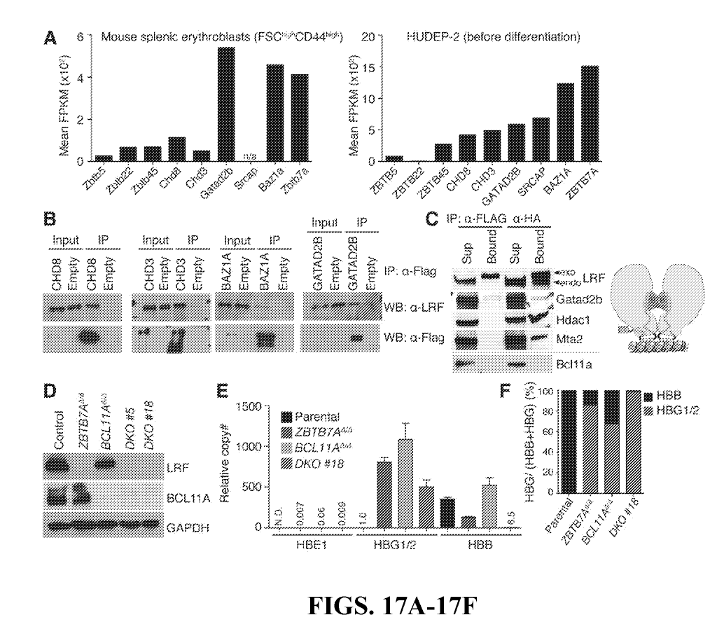

To identify a repressor complex interacting with LRF in an unbiased fashion, Applicants performed a yeast two-hybrid (Y2H) screen with the human LRF-BTB domain as bait. A total of 360 positive clones were processed out of 52.1 million potential binding events. LRF-interacting proteins with high confidence included 4 ZBTB proteins, 3 NuRD/CHD family proteins and two chromatin remodelers (FIGS. 16A and B). Given their abundance in erythroid cells (FIG. 17A) and their potential repressor function, Applicants focused on three factors (GATAD2B, CHD3 and CHD8) for further validation. Interactions of LRF with each were validated by immunoprecipitation (IP) (FIG. 17B). Considering that NuRD complex components are implicated in globin switching (M. Amaya, M. Desai, M. N. Gnanapragasam, S. Z. Wang, et al., Blood 121, 3493-501 (2013); J. Xu, V. G. Sankaran, M. Ni, T. F. Menne, et al., Genes Dev 24, 783-98 (2010); J. W. Rupon, S. Z. Wang, K. Gaensler, J. Lloyd, G. D. Ginder, Proceedings of the National Academy of Sciences 103, 6617-22 (2006), F. C. Costa, H. Fedosyuk, A. M. Chazelle, R. Y. Neades, K. R. Peterson, PLoS Genet 8, e1003155 (2012)), Applicants examined the LRF/GATAD2B interaction in more detail. IP of human proerythroblast lysates with an anti-LRF antibody pulled down GATAD2B and other NuRD complex components (FIG. 5A). The interactions were also validated in mouse erythroid cells (FIG. 15C). Although BCL11A reportedly interacts with NuRD components (V. G. Sankaran, T. F. Menne, J. Xu, T. E. Akie, et al., Science 322, 1839-42 (2008); J. Xu, D. E. Bauer, M. A. Kerenyi, T. D. Vo, et al., Proc Natl Acad Sci USA 110, 6518-23 (2013)), Applicants did not detect BCL11A in the NuRD complexes containing LRF in either human or mouse erythroid cells (FIGS. 5A and 17C). For reciprocal validation, Applicants performed IP in human erythroid cell lysates with anti-GATAD2B or -MTA2 antibody, and as expected, both antibodies pulled-down LRF (FIG. 5B). BCL11A was readily detected in MTA2-containing protein complexes, as reported (J. Xu, D. E. Bauer, M. A. Kerenyi, T. D. Vo, et al., Proc Natl Acad Sci USA 110, 6518-23 (2013)). Consistent with our findings, LRF was not identified as a BCL11A-interacting protein in proteomic affinity screens in erythroid cells (V. G. Sankaran, T. F. Menne, J. Xu, T. E. Akie, et al., Science 322, 1839-42 (2008); J. Xu, D. E. Bauer, M. A. Kerenyi, T. D. Vo, et al., Proc Natl Acad Sci USA 110, 6518-23 (2013)).

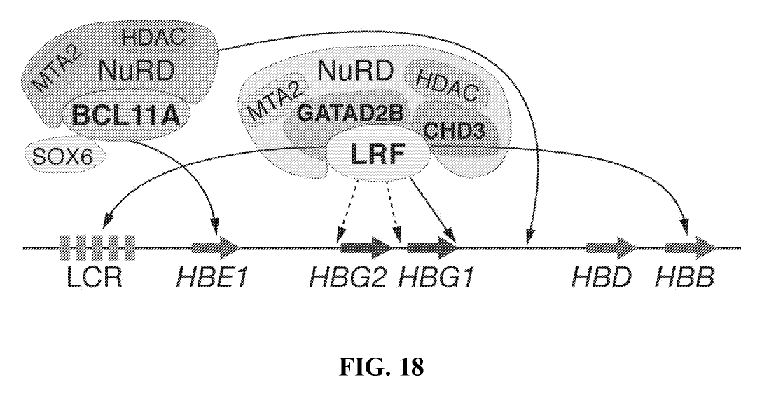

Finally, to determine whether LRF and BCL11A suppress γ-globin expression via distinct mechanisms, Applicants established ZBTB7A/BCL11A double knockout (DKO) HUDEP-2 cells (FIG. 17D) and compared HbF in these cells to that in ZBTB7A- or BCL11A-single KO HUDEP-2 cells. DKO cells exhibited a significantly greater fetal/adult γ-globin ratio than did either ZBTB7A or BCL11A single KO cells (FIGS. 17E and 17F). The HbF levels of DKO cells were at 91 to 94% of total Hb (FIGS. 5C and 5D). These data suggest that LRF and BCL11A represent a primary fetal globin repressive activity in adult erythroid cells (FIG. 18).

Applicants have shown that LRF is a potent repressor of embryonic/fetal γ-like globin expression in adult erythroid cells. Applicants postulate that LRF depletion opens local chromatin at the γ-globin genes, enabling erythroid transcriptional activators to induce γ-globin expression. Furthermore, Applicants propose that LRF silences γ-globin expression independently of BCL11A based on the following observations. First, LRF inactivation in mice specifically reactivates Hbb-bh1, but not Hbb-y, expression, whereas BCL11A depletion induces both of these embryonic globins (J. Xu, C. Peng, V. G. Sankaran, Z. Shao, et al., Science 334, 993-6 (2011)). Second, LRF directly binds to the HBG1 gene, whereas BCL11A reportedly targets intergenic region(s), the LCR, and sequences between HBG1 and HBD (J. Xu, V. G. Sankaran, M. Ni, T. F. Menne, et al., Genes Dev 24, 783-98 (2010); V. G. Sankaran, J. Xu, T. Ragoczy, G. C. Ippolito, et al., Nature 460, 1093-7 (2009)). Third, the LRF-NuRD complex in adult erythroid cells lacks BCL11A. Finally, ZBTB7A/BCL11A DKO HUDEP-2 cells exhibit significantly greater γ-globin expression than did either ZBTB7A or BCL11A single KO cells.

In summary, our work suggests that the two NuRD-associated pathways are responsible for turning off fetal globin expression in order to switch over to adult globin. These findings may enable the development of therapies to turn on fetal globin expression in individuals with human hemoglobinopathies displaying defective adult globin gene expression.

As referred to herein, an “epitope” can be a linear epitope, comprising a sequence of contiguous amino acids, or conformational, formed by the three-dimensional juxtaposition of amino acids and/or glycosyl groups in the tertiary structure of a protein. An epitope can comprise or consist of as few as at least 6 amino acids that are unique to a polypeptide or protein sequence. An epitope can be longer; for example, an epitope can comprise, consist essentially of or consist of at least 6 or at least 8, or at least 10, or at least 12, or at least 15 or at least 20 amino acids.

The invention also encompasses sequences which are homologous the database sequences recited above. In one embodiment, the sequences according to the invention are at least 95% homologous the sequences referred to above. In other embodiments, they are at least 96%, 97%, 98%, 99% or 100% homologous. Homology can be assessed using any suitable sequence alignment technique. Optimal alignment may be detemined with the use of any suitable algorithm for aligning sequences, non-limiting examples of which include the Smith-Waterman algorithm, the Needleman-Tunsch algorithm, algorithms based on the Burrows-Wheeler Transform (e.g. the Burrcnvs Wheeler Aligner), ClustalW, Clustal X, BLAST, Novoalign (Novocraft Technologies, ELAND (Illumina, San Diego, Calif.), SOAP (available at soap.genomics.org.cn), and Maq (available at maq.sourceforge.net).

The invention also comprehends a non-naturally-occurring or engineered B-cell that expresses an antibody of the invention.

The invention also comprehends a vector expressing an antibody or binding fragment thereof of the invention.

Antibodies of the invention may also comprise a label attached thereto and able to be detected, (e.g. the label can be a radioisotope, fluorescent compound, enzyme or enzyme co-factor).

Proteins or polypeptides referred to herein as “recombinant” are proteins or polypeptides produced by the expression of recombinant nucleic acids.

The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced IL 13 binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

To enhance the binding affinity and/or other biological properties of the antibody, the amino acid sequences of the antibody may be mutated. For example, such mutations include deletion, insertion, and/or substitution of amino acid sequence residues of the antibody. An amino acid mutation is made based on the relative similarity of the amino acid side chain substituents, for example, with respect to hydrophobic properties, hydrophilic properties, charges, or sizes. For example, arginine, lysine, and histidine are each a positively charged residue; alanine, glycine, and serine have a similar size; and phenylalanine, tryptophan, and tyrosine have a similar shape. Therefore, based on the considerations described above, arginine, lysine, and histidine may be biological functional equivalents; alanine, glycine, and serine may be biological functional equivalents; and phenylalanine, tryptophan, and tyrosine may be biological functional equivalents.

Amino acid substitution in a protein in which the activity of the molecule is not completely changed is well known in the art. Typical substitutions include Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly substitutions. Considering mutations with biologically equivalent activity, an antibody specifically binding to RAGE or the antigen-binding fragments thereof may also include sequences substantially identical to sequences disclosed herein. In this regard, a substantially identical amino acid sequence may be a sequence with at least 60% homology, at least 70% homology, at least 80% homology, at least 90%, at least 95% homology or 100% homology to a sequence disclosed herein, when the amino acid sequences are aligned to correspond to each other as much as possible. The aligned amino acid sequences are analyzed using an algorithm known in the art. Alignment methods for sequence comparison are well known to one of ordinary skill in the art. For example, a sequence analysis program available on the Internet at the NCBI Basic Local Alignment Search Tool (BLAST) home page, such as blastp, blastx, tblastn, or tblastx, may be used.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an itntnunoglobulin or antibody that binds antigen or competes with intact antibody (i.e., with the intact antibody from which they were derived) for antigen binding (i.e., specific binding).

It is intended that the term “antibody” encompass any Ig class or any Ig subclass (e.g. the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of (IgA1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric form, and IgA antibodies exist in monomeric, dimeric or multimeric form.

The term “IgG subclass” refers to the four subclasses of immunoglobulin class IgG—IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an agent, inhibitor, small molecule, peptide or fragment thereof.

“Specific binding” of an antibody means that the antibody exhibits appreciable affinity for a particular antigen or epitope and, generally, does not exhibit significant crossreactivity. “Appreciable” binding includes binding with an affinity of at least 25 μM. Antibodies with affinities greater than 1×10⁷ M⁻¹ (or a dissociation coefficient of 1 μm or less or a dissociation coefficient of 1 nm or less) typically bind with correspondingly greater specificity. Values intermediate of those set forth herein are also intended to be within the scope of the present invention and antibodies of the invention bind to the domain of interest with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to the domain of interest. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Dose or dosage levels for antibodies in suitable and/or preferred pharmaceutical formulations can be determined in view of the present disclosure and general knowledge of formulation technology, depending upon the intended route of administration, delivery format, and desired dosage. Typically, a physician will determine the actual dosage which will be most suitable for an individual subject.

The specific dose level and frequency of dosage for any particular patient or subject in need of treatment thereof may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. For example, the antibody compound or composition may be administered at a dose of between 2 mg/kg and 0.1 mg/kg, depending on its activity.

The agent, inhibitor, small molecule, peptide or fragment thereof may be administered as a fixed dose, independent of a dose per subject weight ratio, or at an appropriate dose in mg/kg body weight with an approximate maximum of 200 mg/kg. The agent, inhibitor, small molecule, peptide or fragment thereof may be administered to a 70 kg individual in one or more separate, simultaneous or sequential doses of 14,000 mg or less of agent, inhibitor, small molecule, peptide or fragment thereof, 13,000 mg or less of agent, inhibitor, small molecule, peptide or fragment thereof, 12,000 mg or less of agent, inhibitor, small molecule, peptide or fragment thereof, 11,000 mg or less of agent, inhibitor, small molecule, peptide or fragment thereof, 10,000 mg or less of agent, inhibitor, small molecule, peptide or fragment thereof, 9000 mg or less of agent, inhibitor, small molecule, peptide or fragment thereof, 8000 mg or less of agent, inhibitor, small molecule, peptide or fragment thereof, 7000 mg or less of agent, inhibitor, small molecule, peptide or fragment thereof, 6000 mg or less of agent, inhibitor, small molecule, peptide or fragment thereof, 5000 mg or less of agent, inhibitor, small molecule, peptide or fragment thereof, 4500 mg or less of agent, inhibitor, small molecule, peptide or fragment thereof, 4000 mg or less of agent, inhibitor, small molecule, peptide or fragment thereof, 3700 mg or less of agent, inhibitor, small molecule, peptide or fragment thereof, 3500 mg or less of agent, inhibitor, small molecule, peptide or fragment thereof, mg or less of agent, inhibitor, small molecule, peptide or fragment thereof, 3200 mg or less of agent, inhibitor, small molecule, peptide or fragment thereof, 3000 mg or less of agent, inhibitor, small molecule, peptide or fragment thereof, 2700 mg or less of agent, inhibitor, small molecule, peptide or fragment thereof, 2500 mg or less of agent, inhibitor, small molecule, peptide or fragment thereof, 2200 mg or less of agent, inhibitor, small molecule, peptide or fragment thereof, or 2100 mg or less of agent, inhibitor, small molecule, peptide or fragment thereof, 1700 mg or less of agent, inhibitor, small molecule, peptide or fragment thereof, 1500 mg or less of agent, inhibitor, small molecule, peptide or fragment thereof, 1200 mg or less of agent, inhibitor, small molecule, peptide or fragment thereof, or 1100 mg or less of agent, inhibitor, small molecule, peptide or fragment thereof, 1000 mg or less of agent, inhibitor, small molecule, peptide or fragment thereof, 700 mg or less of agent, inhibitor, small molecule, peptide or fragment thereof, 500 mg or less of agent, inhibitor, small molecule, peptide or fragment thereof, 200 mg or less of agent, inhibitor, small molecule, peptide or fragment thereof, or 100 mg or less of agent, inhibitor, small molecule, peptide or fragment thereof. In another embodiment, the agent, inhibitor, small molecule, peptide or fragment is administered in one or more doses of at least 20 mg of agent, inhibitor, small molecule, peptide or fragment thereof. Since the total protein concentration in human plasma is 70,000 mg/l, and standard blood transfusion or plasma or albumin infusions routinely deliver tens or even hundreds of grams of protein intravenously, administration of the maximal doses of anti-sickle cell anemia or β-thalassemia are safe and acceptable.

The of anti-sickle cell anemia or β-thalassemia compound or composition may be administered as a fixed dose, independent of a dose per subject weight ratio. The of anti-sickle cell anemia or β-thalassemia compound or composition may be administered in one or more separate, simultaneous or sequential parenteral doses of 100 mg or less, of 50 mg or less, 25 mg or less, or 10 mg or less. Alternatively, of anti-sickle cell anemia or β-thalassemia compound or composition may be administered in a dose per subject weight ratio as easily determined by one of skill in the art. The component(s) may be formulated into a pharmaceutical composition, such as by mixing with one or more of a suitable carrier, diluent or excipient, by using techniques that are known in the art.

Vector Delivery Systems

One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

Preferably, delivery is in the form of a vector which may be a viral vector, such as a lenti- or baculo- or preferably adeno-viral/adeno-associated viral vectors, but other means of delivery are known (such as yeast systems, microvesicles, gene guns/means of attaching vectors to gold nanoparticles) and are provided. A vector may mean not only a viral or yeast system (for instance, where the nucleic acids of interest may be operably linked to and under the control of (in terms of expression, such as to ultimately provide a processed RNA) a promoter), but also direct delivery of nucleic acids into a host cell. While in herein methods the vector may be a viral vector and this is advantageously an AAV, other viral vectors as herein discussed can be employed, such as lentivirus. For example, baculoviruses may be used for expression in insect cells. These insect cells may, in turn be useful for producing large quantities of further vectors, such as AAV or lentivirus vectors adapted for delivery of the present invention. Also envisaged is a method of delivering the present compositions comprising nucleic acids encoding anti-RAGE antibodies of the invention comprising delivering to a cell mRNA encoding said anti-RAGE antibodies. AAV and lentiviral vectors are preferred. Delivering a non-naturally occurring or engineered composition comprising an AAV or lentivirus vector system comprising one or more AAV or lentivirus vectors operably encoding a composition for expression thereof.

Use of viral vectors for antibody-based protection (commonly referred to as “vectored immunprophylaxis”) is known in the art. For example, Balzas et al. discuss antibody-based protection against HIV infection by vectored immunoprophylaxis (see e.g., Balazs A. B. et al. Nature 2011 Nov. 30; 481(7379):81-4. Antibody-based protection against HIV infection by vectored immunoprophylaxis; Balazs A. B. et al. Nat. Med. 2014 March; 20(3):296-300. Vectored immunoprophylaxis protects humanized mice from mucosal HIV transmission.).

The invention in some embodiments comprehends a method of preparing the AAV of the invention comprising transfecting plasmid(s) containing or consisting essentially of nucleic acid molecule(s) coding for the AAV into AAV-infected cells, and supplying AAV rep and/or cap obligatory for replication and packaging of the AAV. In some embodiments the AAV rep and/or cap obligatory for replication and packaging of the AAV are supplied by transfecting the cells with helper plasmid(s) or helper virus(es). In some embodiments the helper virus is a poxvirus, adenovirus, herpesvirus or baculovirus. In some embodiments the poxvirus is a vaccinia virus. In some embodiments the cells are mammalian cells. And in some embodiments the cells are insect cells and the helper virus is baculovirus. In other embodiments, the virus is a lentivirus.

As to AAV, the AAV can be AAV1, AAV2, AAV5 or any combination thereof. One can select the AAV of the AAV with regard to the cells to be targeted; e.g., one can select AAV serotypes 1, 2, 5 or a hybrid or capsid AAV1, AAV2, AAV5 or any combination thereof for targeting brain or neuronal cells; and one can select AAV4 for targeting cardiac tissue. AAV8 is useful for delivery to the liver. The above promoters and vectors are preferred individually. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No 5,846,946 and as in clinical studies involving plasmids. Doses may be based on or extrapolated to an average 70 kg individual, and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed.

The viral vectors can be injected into the tissue of interest. The expression of anti-RAGE antibodies can be driven by a cell-type specific promoter. For example, liver-specific expression might use the Albumin promoter and neuron-specific expression might use the Synapsin I promoter.

In some embodiments, the viral vector is delivered to the tissue of interest by, for example, an intramuscular injection, while other times the viral delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector chose, the target cell, organism, or tissue, the general condition of the subject to be treated, the degree of transformation/modification sought, the administration route, the administration mode, the type of transformation/modification sought, etc.

Such a dosage may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, an adjuvant to enhance antigenicity, an immunostimulatory compound or molecule, and/or other compounds known in the art. The adjuvant herein may contain a suspension of minerals (alum, aluminum hydroxide, aluminum phosphate) on which antigen is adsorbed; or water-in-oil emulsion in which antigen solution is emulsified in oil (MF-59, Freund's incomplete adjuvant), sometimes with the inclusion of killed mycobacteria (Freund's complete adjuvant) to further enhance antigenicity (inhibits degradation of antigen and/or causes influx of macrophages). Adjuvants also include immunostimulatory molecules, such as cytokines, costimulatory molecules, and for example, immunostimulatory DNA or RNA molecules, such as CpG oligonucleotides. Such a dosage formulation is readily ascertainable by one skilled in the art. The dosage may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

In an embodiment herein the delivery is via an adenovirus, which may be at a single booster dose containing at least 1×10⁵ particles (also referred to as particle units, pu) of adenoviral vector. In an embodiment herein, the dose preferably is at least about 1×10⁶ particles (for example, about 1×10⁶-1×10¹² particles), more preferably at least about 1×10⁷ particles, more preferably at least about 1×10⁸ particles (e.g., about 1×10⁸-1×10¹¹ particles or about 1×10⁸-1×10¹² particles), and most preferably at least about 1×10⁹ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ articles). Thus, the dose may contain a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In an embodiment herein, the delivery is via an AAV. A therapeutically effective dosage for in vivo delivery of the AAV to a human is believed to be in the range of from about 20 to about 50 ml of saline solution containing from about 1×10¹⁰ to about 1×10¹⁰ functional AAV/ml solution. The dosage may be adjusted to balance the therapeutic benefit against any side effects. In an embodiment herein, the AAV dose is generally in the range of concentrations of from about 1×10⁵ to 1×10⁵⁰ genomes AAV, from about 1×10⁸ to 1×10²⁰ genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

In an embodiment herein the delivery is via a plasmid. In such plasmid compositions, the dosage should be a sufficient amount of plasmid to elicit a response. For instance, suitable quantities of plasmid DNA in plasmid compositions can be from about 0.1 to about 2 mg, or from about 1 μg to about 10 μg.

The doses herein are based on an average 70 kg individual. The frequency of administration is within the ambit of the medical or veterinary practitioner (e.g., physician, veterinarian), or scientist skilled in the art.

### Lentivirus

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses may be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media was changed to OptiMiEM (serum-free) media and transfection was done 4 hours later. Cells were transfected with 10 μg of lentiviral transfer plasmid (pCasES10) and the following packaging plasmids: 5 μg of pMD2.G (VSV-g pseudotype), and 7.5 ug of psPAX2 (gag/pol/rev/tat). Transfection was done in 4 mL OptiMEM with a cationic lipid delivery agent (50 μL Lipofectamine 2000 and 100 μl Plus reagent). After 6 hours, the media was changed to antibiotic-free DMEM with 10% fetal bovine serum. Lentivirus may be purified as follows. Viral supernatants were harvested after 48 hours. Supernatants were first cleared of debris and filtered through a 0.45 um low protein binding (PVDF) filter. They were then spun in a ultracentrifuge for 2 hours at 24,000 rpm. Viral pellets were resuspended in 50 μl of DMEM overnight at 4 C. They were then aliquotted and immediately frozen at −80 C.

In another embodiment, minimal non-primate lentiviral vectors based on the equine infectious anemia virus (EIAV) are also contemplated, especially for ocular gene therapy (see, e.g., Balagaan, J Gene Med 2006; 8: 275-285, Published online 21 Nov. 2005 in Wiley InterScience (www.interscience.wiley.com). DOI: 10.1002/jgm.845). In another embodiment, RetinoStat®, an equine infectious anemia virus-based lentiviral gene therapy vector that expresses angiostatic proteins endostain and angiostatin that is delivered via a subretinal injection for the treatment of the web form of age-related macular degeneration is also contemplated (see, e.g., Binley et al., HUMAN GENE THERAPY 23:980-991 (September 2012)) may be modified for expressing antibody(ies) of the present invention.

In another embodiment, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted for expressing antibody(ies) of the present invention. A minimum of 2.5×106 CD34+ cells per kilogram patient weight may be collected and prestimulated for 16 to 20 hours in X-VIVO 15 medium (Lonza) containing 2 mM L-glutamine, stem cell factor (100 ng/ml), Flt-3 ligand (Flt-3L) (100 ng/ml), and thrombopoietin (10 ng/ml) (CellGenix) at a density of 2×106 cells/ml. Prestimulated cells may be transduced with lentiviral at a multiplicity of infection of 5 for 16 to 24 hours in 75-cm2 tissue culture flasks coated with fibronectin (25 mg/cm2) (RetroNectin, Takara Bio Inc.).

Lentiviral vectors have been disclosed as in the treatment for Parkinson's Disease, see, e.g., US Patent Publication No. 20120295960 and U.S. Pat. Nos. 7,303,910 and 7,351,585, and for delivery of anti-αβ antibodies for the treatment of Alzheimers disease (Fukuchi et al., Neurobiol Dis. 2006 September; 23(3): 502-511). Lentiviral vectors have also been disclosed for the treatment of ocular diseases, see e.g., US Patent Publication Nos. 20060281180, 20090007284, US20110117189; US2009001543; US20070054961, US20100317109. Lentiviral vectors have also been disclosed for delivery to the brain, see, e.g., US Patent Publication Nos. US20110293571, US20110293571, US20040013648, US20070025970, US20090111106 and U.S. Pat. No. 7,259,015. These vectors can be adapted to express the antibody(ies) of the invention, and can be administered in analogous amounts, albeit advantageously iv or im or to the tumor mass rather than to the brain or eye (unless, of course, the tumor is in the brain or eye).

In certain embodiments, the assay to determine the characteristics of cells is selected in a manner appropriate to the cell type and agent and/or environmental factor being studied as disclosed in WO 2002/04113, which is hereby incorporated by reference in its entirely. For example, changes in cell morphology may be assayed by standard light, or electron microscopy. Alternatively, the effects of treatments or compounds potentially affecting the expression of cell surface proteins may be assayed by exposing the cells to either fluorescently labeled ligands of the proteins or antibodies to the proteins and then measuring the fluorescent emissions associated with each cell on the plate. As another example, the effects of treatments or compounds which potentially alter the pH or levels of various ions within cells may be assayed using various dyes which change in color at determined pH values or in the presence of particular ions. The use of such dyes is well known in the art. For cells, which have been transformed or transfected with a genetic marker, such as the β-galactosidase, alkaline phosphatase, or luciferase genes, the effects of treatments or compounds may be assessed by assays for expression of that marker. In particular, the marker may be chosen so as to cause spectrophotometrically assayable changes associated with its expression.

Particular screening applications of this invention relate to the testing of pharmaceutical compounds in drug research. The reader is referred generally to the standard textbook In vitro Methods in Pharmaceutical Research, Academic Press, 1997, and U.S. Pat. No. 5,030,015. In certain aspects of this invention, the culture of the invention is used to grow and differentiate a target cell to play the role of test cells for standard drug screening and toxicity assays. Assessment of the activity of candidate pharmaceutical compounds generally involves combining the target cell (e.g., a myocyte, an adipocyte or a hepatocyte) with the candidate compound, determining any change in the morphology, marker phenotype, or metabolic activity of the cells that is attributable to the candidate compound (compared with untreated cells or cells treated with an inert compound, such as vehicle), and then correlating the effect of the candidate compound with the observed change. The screening may be done because the candidate compound is designed to have a pharmacological effect on the target cell, or because a candidate compound may have unintended side effects on the target cell. Alternatively, libraries can be screened without any predetermined expectations in hopes of identifying compounds with desired effects.

Cytotoxicity can be determined in the first instance by the effect on cell viability and morphology. In certain embodiments, toxicity may be assessed by observation of vital staining techniques, ELISA assays, immunohistochemistry, and the like or by analyzing the cellular content of the culture, e.g., by total cell counts, and differential cell counts or by metabolic markers such as MTT and XTT.

Additional further uses of the culture of the invention include, but are not limited to, its use in research e.g., to elucidate hemoglobinopathy mechanisms leading to the identification of novel targets for hemoglobinopathy therapies, and to generate genotype-specific cells for disease modeling, including the generation of new therapies customized to different genotypes. Such customization can reduce adverse drug effects and help identify therapies appropriate to the patient's genotype.

The present invention further provides a method for monitoring a patient with a hemoglobinopathy (e.g., sickle cell anemia or β-thalassemia), comprising (i) obtaining a target cell from the patient; (ii) determining the gene expression profile of that cell; and (iii) comparing the transcriptional profile of that target cell and the transcriptional profile of a sickle cell anemia or β-thalassemia cell, wherein the cell type of the target cell and the sickle cell anemia or β-thalassemia cell is the same. Monitoring the sickle cell anemia or β-thalassemia transcriptional profile of a patient is useful, for example, to determine the patient's pharmacological response to a drug or disease progression.

Biopsy refers to the removal of a sample of tissue for purposes of diagnosis. For example, a biopsy is from a muscle, fat, a cancer or tumor, including a sample of tissue from an abnormal area or an entire tumor.

The disclosed methods involve comparing the presence or levels of the disclosed markers in a sample from a subject identified as having a hemoglobinopathy condition to the levels of the same markers in a reference (e.g., levels present in a corresponding sample from a healthy control). It is understood a reference includes a concurrently run control, or a standard created by assaying one or more non-hemoglobinopathy cells and collecting the marker data. Thus, the control sample is optionally a standard that is created and used continuously. The standard includes, for example, the average level of a biomarker in a sample from a non-hemoglobinopathy control group.

Also provided is a method of predicting or monitoring the efficacy of an anti-sickle cell anemia or β-thalassemia agent in a subject. The method comprises acquiring a biological sample, such as tissue or bodily fluid, from the subject after administering the agent to the subject. For example, the tissue or bodily fluid is collected from the subject 1 to 60 minutes, hours, days, or weeks after administering the agent to the subject.

In diagnostics, capture arrays are used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, optionally in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods are automated for high throughput production. For capture arrays and protein function analysis, it is important that proteins be correctly folded and functional; this is not always the case, e.g., where recombinant proteins are extracted from bacteria under denaturing conditions. Nevertheless, arrays of denatured proteins are useful in screening antibodies for cross-reactivity, and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, N.J.) and specialized chip designs, such as engineered microchannels in a plate (e.g., The Living Chip™, Biotrove, Woburn, Mass.) and tiny 3D posts on a silicon surface (Zyomyx, Hayward Calif.). Particles in suspension are also used as the basis of arrays, providing they are coded for identification; systems include color coding for microbeads (Luminex, Austin, Tex.; Bio-Rad Laboratories), semiconductor nanocrystals (e.g., QDOTS™, Quantum Dot, Hayward, Calif.), barcoding for beads (ULTRAPLEK™ beads, SmartBead Technologies Ltd, Babraham, Cambridge, UK) and multimetal microrods (e.g., NANOBARCODES™ particles, Nanoplex Technologies, Mountain View, Calif.). Beads are optionally assembled into planar arrays on semiconductor chips (LEAPS™ technology, BioArray Solutions, Warren, N.J.).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control. It may or may not alter the functional properties of the protein, and reproducibility and efficiency are variable. Covalent coupling methods provide a stable linkage, are applied to a range of proteins and have good reproducibility. However, orientation is variable. Furthermore, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately, and the array may require special handling and have variable stability.

Several immobilization chemistries and tags have been described for fabrication of protein arrays. Substrates for covalent attachment include glass slides coated with amino- or aldehyde-containing silane reagents. In the VERSALINX™ system (Prolinx, Bothell, Wash.) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HYDROGEL™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilized on a surface such as titanium dioxide (Zyomyx, Inc., Hayward, Calif.) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences, Affymetrix Inc. and Genetix] as well as manual equipment [e.g., V & P Scientific]. Bacterial colonies are optionally robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Nanosciences Inc. and Nanolink Inc., for example, have developed commercially available nanoarrays.

Fluorescence labeling and detection methods are widely used. The same instrumentation as used for reading DNA microarrays is applicable to protein arrays. For differential display, capture (e.g., antibody) arrays are probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity is amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity is achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, Ariz.), rolling circle DNA amplification (Molecular Staging, New Haven, Conn.), mass spectrometry (Intrinsic Bioprobes; Ciphergen, Fremont, Calif.), resonance light scattering (Genicon Sciences, San Diego, Calif.) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, Calif.; Clontech, Mountain View, Calif.; BioRad; Sigma, St. Louis, Mo.). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; BioInvent, Lund, Sweden; Affitech, Walnut Creek, Calif.; Biosite, San Diego, Calif.). In addition to the conventional antibodies, Fab and say fragments, single V-domains from camelids or engineered human equivalents (Domantis, Waltham, Mass.) are optionally useful in arrays.

The term scaffold refers to ligand-binding domains of proteins, which are engineered into multiple variants capable of binding diverse target molecules with antibody-like properties of specificity and affinity. The variants are produced in a genetic library format and selected against individual targets by phage, bacterial or ribosome display. Such ligand-binding scaffolds or frameworks include Affibodies based on S. aureus protein A (Affibody, Bromma, Sweden), Trinectins based on fibronectins (Phylos, Lexington, Mass.) and Anticalins based on the lipocalin structure (Pieris Proteolab, Freising-Weihenstephan, Germany). These are used on capture arrays in a similar fashion to antibodies and have advantages of robustness and ease of production.

Nonprotein capture molecules, notably the single-stranded nucleic acid aptamers which bind protein ligands with high specificity and affinity, are also used in arrays (SomaLogic, Boulder, Colo.). Aptamers are selected from libraries of oligonucleotides by the Selex™ procedure (SomaLogic, Boulder, Colo.) and their interaction with protein is enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains are used to detect binding.

Protein analytes binding to antibody arrays are detected directly or indirectly, for example, via a secondary antibody. Direct labeling is used for comparison of different samples with different colors. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately. Serial dilution of the sample or use of antibodies of different affinities are solutions to this problem. Proteins of interest are frequently those in low concentration in body fluids and extracts, requiring detection in the pictogram (pg) range or lower, such as cytokines or the low expression products in cells.

An alternative to an array of capture molecules is one made through molecular imprinting technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

Another methodology which is useful diagnostically and in expression profiling is the ProteinChip® array (Ciphergen, Fremont, Calif.), in which solid phase chromatographic surfaces bind proteins with similar characteristics of charge or hydrophobicity from mixtures such as plasma or tumor extracts, and SELDI-TOF mass spectrometry is used to detection the retained proteins.

Large-scale functional chips have been constructed by immobilizing large numbers of purified proteins and are used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g., via a His tag and immobilized. Cell free protein transcription/translation is a viable alternative for synthesis of proteins which do not express well in bacterial or other in vivo systems.

For detecting protein-protein interactions, protein arrays are in vitro alternatives to the cell-based yeast two-hybrid system and are useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilized on a microarray. Large-scale proteome chips are also useful in identification of functional interactions, drug screening, etc. (Proteometrix, Branford, Conn.).

As a two-dimensional display of individual elements, a protein array is used to screen phage or ribosome display libraries, in order to select specific binding partners, including antibodies, synthetic scaffolds, peptides and aptamers. In this way, library against library screening is carried out. Screening of drug candidates in combinatorial chemical libraries against an array of protein targets identified from genome projects is another application of the approach.

Multiplexed bead assays use a series of spectrally discrete particles that are used to capture and quantitate soluble analytes. The analyte is then measured by detection of a fluorescence-based emission and flow cytometric analysis. Multiplexed bead assays generate data that is comparable to ELISA based assays, but in a multiplexed or simultaneous fashion. Concentration of unknowns is calculated for the cytometric bead array as with any sandwich format assay, i.e., through the use of known standards and by plotting unknowns against a standard curve. Further, multiplexed bead assays allow quantification of soluble analytes in samples never previously considered due to sample volume limitations. In addition to the quantitative data, powerful visual images are generated revealing unique profiles or signatures that provide the user with additional information at a glance.

In some examples of the disclosed methods, when the level of expression of a biomarker(s) is assessed, the level is compared with the level of expression of the biomarker(s) in a reference standard. By reference standard is meant the level of expression of a particular biomarker(s) from a sample or subject lacking a cancer, at a selected stage of cancer, or in the absence of a particular variable such as a therapeutic agent. Alternatively, the reference standard comprises a known amount of biomarker. Such a known amount correlates with an average level of subjects lacking a cancer, at a selected stage of cancer, or in the absence of a particular variable such as a therapeutic agent. A reference standard also includes the expression level of one or more biomarkers from one or more selected samples or subjects as described herein. For example, a reference standard includes an assessment of the expression level of one or more biomarkers in a sample from a subject that does not have a cancer, is at a selected stage of progression of a cancer, or has not received treatment for a cancer. Another exemplary reference standard includes an assessment of the expression level of one or more biomarkers in samples taken from multiple subjects that do not have a cancer, are at a selected stage of progression of a cancer, or have not received treatment for a cancer.

When the reference standard includes the level of expression of one or more biomarkers in a sample or subject in the absence of a therapeutic agent, the control sample or subject is optionally the same sample or subject to be tested before or after treatment with a therapeutic agent or is a selected sample or subject in the absence of the therapeutic agent. Alternatively, a reference standard is an average expression level calculated from a number of subjects without a particular cancer. A reference standard also includes a known control level or value known in the art. In one aspect of the methods disclosed herein, it is desirable to age-match a reference standard with the subject diagnosed with a cancer.

In one technique to compare protein levels of expression from two different samples (e.g., a sample from a subject diagnosed with a cancer and a reference standard), each sample is separately subjected to 2D gel electrophoresis. Alternatively, each sample is differently labeled and both samples are loaded onto the same 2D gel. See, e.g., Unlu et al. Electrophoresis, 1997; 18:2071-2077, which is incorporated by reference herein for at least its teachings of methods to assess and compare levels of protein expression. The same protein or group of proteins in each sample is identified by the relative position within the pattern of proteins resolved by 2D electrophoresis. The expression levels of one or more proteins in a first sample is then compared to the expression level of the same protein(s) in the second sample, thereby allowing the identification of a protein or group of proteins that is expressed differently between the two samples (e.g., a biomarker). This comparison is made for subjects before and after they are suspected of having a cancer, before and after they begin a therapeutic regimen, and over the course of that regimen.

In another technique, the expression level of one or more proteins is in a single sample as a percentage of total expressed proteins. This assessed level of expression is compared to a preexisting reference standard, thereby allowing for the identification of proteins that are differentially expressed in the sample relative to the reference standard.

There are a variety of sequences related to biomarkers as well as any other protein disclosed herein that are disclosed on GenBank, and these sequences and others are herein incorporated by reference in their entireties as well as for individual subsequences contained therein. Thus, a variety of sequences are provided herein and these and others are found in GenBank at http://ww.ncbi.nih.gov/entrez/query.fcgi. Those of skill in the art understand how to resolve sequence discrepancies and differences and to adjust the compositions and methods relating to a particular sequence to other related sequences. Primers and/or probes are designed for any sequence given the information disclosed herein and known in the art.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant invention, reference is made to: U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641; US Patent Publication US 2015-0031134 (U.S. application Ser. No. 14/497,627), which is allowed; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US 2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809), WO 2015/089351 (PCT/US2014/069897), WO 2015/089354 (PCT/US2014/069902), WO 2015/089364 (PCT/US2014/069925), WO 2015/089427 (PCT/US2014/070068), WO 2015/089462 (PCT/US2014/070127), WO 2015/089419 (PCT/US2014/070057), WO 2015/089465 (PCT/US2014/070135), WO 2015/089486 (PCT/US2014/070175), PCT/US2015/051691, PCT/US2015/051830. Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/835,973, 61/836,080, 61/836,101, and 61/836,127, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT/US2014/62558 filed Oct. 28, 2014, and U.S. Provisional Patent Applications Ser. Nos. 61/915,148, 61/915,150, 61/915,153, 61/915,203, 61/915,251, 61/915,301, 61/915,267, 61/915,260, and 61/915,397, each filed Dec. 12, 2013; 61/757,972 and 61/768,959, filed on Jan. 29, 2013 and Feb. 25, 2013; 62/010,888 and 62/010,879, both filed Jun. 11, 2014; 62/010,329, 62/010,439 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/055,484, 62/055,460 and 62/055,487, each filed Sep. 25, 2014; and 62/069,243, filed Oct. 27, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to PCT application designating, inter alia, the United States, application No. PCT/US14/41806, filed Jun. 10, 2014.

Mention is also made of U.S. application 62/180,709, 17 Jun. 2015, PROTECTED GUIDE RNAS (PGRNAS); U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. applications 62/091,462, 12 Dec. 2014, 62/096,324, 23 Dec. 2014, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 2014 and 62/180,692, 17 Jun. 2015, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, 62/181,641, 18 Jun. 2015, and 62/181,667, 18 Jun. 2015, RNA-TARGETING SYSTEM; U.S. application 62/096,656, 24 Dec. 2014 and 62/181,151, 17 Jun. 2015, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAT REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12 Feb. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 2014 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, and Attorney Docket No. 46783.01.2128, filed 22 Oct. 2015, NOVEL CRISPR ENZYMES AND SYSTEMS, U.S. application 62/232,067, 24 Sep. 2015, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

Also with respect to general information on CRISPR-Cas Systems, mention is made of the following (also hereby incorporated herein by reference):
Multiplex genuine engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. 2013 Aug. 22; 500(7463):472-6. doi: 10.1038/Nature12466. Epub 2013 Aug. 23;
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5. (2013);
DNA targeting specfcity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308. (2013);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013). [Epub ahead of print];
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27. (2014). 156(5):935-49;
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. (2014) April 20. doi: 10.1038/nbt.2889,
CRISPR-Cas9 Knockin Mice for Genome Editing and Cancer Modeling, Platt et al., Cell 159(2): 440-455 (2014) DOI: 10.1016/j.cell.2014.09.014,
Development and Applications of CRISPR-Cas9 for Genome Engineering, Hsu et al, Cell 157, 1262-1278 (Jun. 5, 2014) (Hsu 2014),
Genetic screens in human cells using the CRISPR/Cas9 system, Wang et al., Science. 2014 Jan. 3; 343(6166): 80-84. doi:10.1126/science.1246981,
Rational design of highly active sgRNAs for CRISPR-Cas9-mediated gene inactivation, Doench et al., Nature Biotechnology published online 3 Sep. 2014; doi:10.1038/nbt.3026, and
In vivo interrogation of gene function in the mammalian brain using CRISPR-Cas9, Swiech et al, Nature Biotechnology; published online 19 Oct. 2014; doi:10.1038/nbt.3055.
Genome-scale transcriptional activation by an engineered CRISPR-Cas9 complex, Konermann S, Brigham M D, Trevino A E, Joung J, Abudayyeh O O, Barcena C, Hsu, P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91 (2015).
High-throughput functional genomics using CRISPR-Cas9, Shalem et al., Nature Reviews Genetics 16, 299-311 (May 2015).
Sequence determinants of improved CRISPR sgRNA design, Xu et al., Genome Research 25, 1147-1157 (August 2015).
A Genome-wide CRISPR Screen in Primary Immune Cells to Dissect Regulatory Networks, Parnas et al., Cell 162, 675-686 (Jul. 30, 2015).
CRISPR/Cas9 cleavage of viral DNA efficiently suppresses hepatitis B virus, Ramanan et al., Scientific Reports 5:10833. doi: 10.1038/srep10833 (Jun. 2, 2015).
Crystal Structure of Staphylococcus aureus Cas9, Nishimasu et al., Cell 162, 1113-1126 (Aug. 27, 2015).
BCL11A enhancer dissection by Cas9-mediated in situ saturating mutagenesis, Canver et al., Nature 527(7577):192-7 (Nov. 12, 2015) doi: 10.1038/nature15521. Epub 2015 Sep. 16.
Cpf1 Is a Single RNA-Guided Endonuclease Class 2 CRISPR-Cas System, Zetsche et al., Cell 163, 759-71 (Sep. 25, 2015).
Discovery and Functional Characterization of Diverse Class 2 CRISPR-Cas Systems, Shmakov et al., Molecular Cell, 60(3), 385-397 doi: 10.1016/j.molcel.2015.10.008 Epub Oct. 22, 2015.
Rationally engineered Cas9 nucleases with improved specificity, Slaymaker et al., Science 2015 Dec. 1. pii: aad5227. [Epub ahead of print]
   each of which is incorporated herein by reference, and discussed briefly below:
Cong et al. engineered type II CRISPR/Cas systems for use in eukaryotic cells based on both Streptococcus thermophilus Cas9 and also Streptoccocus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR/Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR/Cas system for the one-step generation of mice carrying mutations in multiple genes which were traditionally generated in multiple steps by sequential recombination in embryonic stem cells and/or time-consuming intercrossing of mice with a single mutation. The CRISPR/Cas system will greatly accelerate the in vivo study of functionally redundant genes and of epistatic gene interactions.
Konermann et al. addressed the need in the art for versatile and robust technologies that enable optical and chemical modulation of DNA-binding domains based CRISPR Cas9 enzyme and also Transcriptional Activator Like Effectors.
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and sgRNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays. Shalem et al. (2015) described ways in which catalytically inactive Cas9 (dCas9) fusions are used to synthetically repress (CRISPRi) or activate (CRISPRa) expression, showing advances using Cas9 for genome-scale screens, including arrayed and pooled screens, knockout approaches that inactivate genomic loci and strategies that modulate transcriptional activity.
Shalem et al. (2015) described ways in which catalytically inactive Cas9 (dCas9) fusions are used to synthetically repress (CRISPRi) or activate (CRISPRa) expression, showing advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR/Cas9 knockout and nucleotide preference at the cleavage site. The authors also found that the sequence preference for CRISPRi/a is substantially different from that for CRISPR/Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-ITGGGT-3′ PAM. A structural comparison of SaCas9 with SpCas9 highlighted both structural conservation and divergence, explaining their distinct PAM specificities and orthologous sgRNA recognition.
Canver et al. (2015) demonstrated a CRISPR-Cas9-based functional investigation of non-coding genomic elements. The authors we developed pooled CRISPR-Cas9 guide RNA libraries to perform in situ saturating mutagenesis of the human and mouse BCL11A enhancers which revealed critical features of the enhancers.
Zetsche et al. (2015) reported characterization of Cpf1, a class 2 CRISPR nuclease from Francisella novicida U112 having features distinct from Cas9. Cpf1 is a single RNA-guided endonuclease lacking tracrRNA, utilizes a T-rich protospacer-adjacent motif, and cleaves DNA via a staggered DNA double-stranded break.
Shmakov et al. (2015) reported three distinct Class 2 CRISPR-Cas systems. Two system CRISPR enzymes (C2c1 and C2c3) contain RuvC-like endonuclease domains distantly related to Cpf1. Unlike Cpf1, C2c1 depends on both crRNA and tracrRNA for DNA cleavage. The third enzyme (C2c2) contains two predicted HEPN RNase domains and is tracrRNA independent.
Slaymaker et al (2015) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.

Mention is also made of Tsai et al, “Dimeric CRISPR RNA-guided FokI nucleases for highly specific genome editing,” Nature Biotechnology 32(6): 569-77 (2014) which is not believed to be prior art to the instant invention or application, but which may be considered in the practice of the instant invention. Mention is also made of Konermann et al., “Genome-scale transcription activation by an engineered CRISPR-Cas9 complex,” doi:10.1038/nature14136, incorporated herein by reference.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction” (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

Targeted Approach: Protein-Fragment Complementation Assay (PCA)

A Y2H screen identified CHD3 aa1181-1378 as the CHD3/LRF-BTB binding domain (CHD3-LBD). Applicants hypothesized that overexpression of CHD3-LBD competitively inhibits endogenous LRF/CHD3 binding, unlocking γ-globin silencing. Applicants determine the minimal interaction fragment in the CHD3-LBD. To do so, a series of N- and C-terminal deletion mutants of the CHD3-LBD are generated and the binding of these fragments to the LRF-BTB domain are assessed by an in vitro binding assay. Applicants validate the relative strengths of interactions between the CHD3-LBD fragments and the LRF-BTB domain by surface plasmon resonance (SPR) biosensor measurements as described (Ahmad K F, Melnick A, Lax S, Bouchard D, Liu J, et al. Mechanism of SMRT corepressor recruitment by the BCL6 BTB domain. Mol Cell. 2003; 12:1551-1564). The minimal peptide fragment within the CHD3-LBD is identified, and the crystal structure of the LRF-BTB/CHD3-LBD minimal fragment complex provided to gain insight into the structural basis of LRF/CHD3-mediated transcriptional repression. Finally, Applicants test whether the CHD3-LBD minimal fragment can competitively interfere with endogenous LRF/CHD3 binding and depress γ-globin expression in HUDEP-2 cells, and thus generate a lentiviral vector encoding a CHD3-LBD minimal fragment fused with the pTAT motif, a protein transduction domain (PTD) derived from the HIV TAT (CHD3-LBDTAT) (Nagahara H, Vocero-Akbani A M, Snyder E L, Ho A, Latham D G, et al. Transduction of full-length TAT fusion proteins into mammalian cells: TAT-p27Kip1 induces cell migration. Nat Med. 1998; 4:1449-1452). Applicants transduce CHD3-LBDTAT into HUDEP-2 cells and determine the levels of γ-globin/HbF expression. In addition, HUDEP-2 cells are incubated with the synthetic CHD3-LBDTAT peptides and γ-globin/HbF levels which are analyzed by q-PCR and HPLC.

Mapping of LRF-BTB/CHD3-LBD Minimal Fragment Complex

Applicants performed computer-aided drug design based on the crystal structure of the LRT-BTB/CHD3-LBD minimal fragment complex (including LRF-BTB and CHD protein binding) (Kelley L. A; Mezulis, S.; Yates, C. M.; Wass, M. N.; Sternberg, M. J. E. “The Phyre2 Web Portal for Protein Modeling, Prediction and analysis” Nature Protocols 2015, 10, 845-858).

SEQ ID NO. 1:
MAGGVDGPIGIPFPDHSSDILSGLNEQRTQGLLCDVVILVEGREFPTH
RSVLAACSQYFKKLFTSGAVVDQQNVYEIDFVSAEALTALMDFAYTAT
LTVSTANVGDILSAARLLEIPAVSHVCADLLDRQI

from Human LRF/ZBTB7A:

MAGGVDGPIGIPFPDHSSDILSGLNEQRTQGLLCDVVILVEGREFPTH
RSVLAACSQYFKKLFTSGAVVDQQNVYEIDFVSAEALTALMDFAYTAT
LTVSTANVGDILSAARLLEIPAVSHVCADLLDRQILAASAGADAGQLD
LVDQIDQRNLLRAKEYLEFFQSNPMNSLPPAAAAAAASFPWSAFGASD
DDLDATKEAVAAAVAAVAAGDCNGLDFYGPGPPAERPPTGDGDEGDSN
PGLWPERDEDAPTGGLFPPPVAPPAATQNGHYGRGGEEEAASLSEAAP
EPGDSPGFLSGAAEGEDGDGPDVDGLAASTLLQQMMSSVGRAGAAAGD
SDEESRADDKGVMDYYLKYFSGAHDGDVYPAWSQKVEKKIRAKAFQKC
PICEKVIQGAGKLPRHIRTHTGEKPYECNICKVRFTRQDKLKVHMRKH
TGEKPYLCQQCGAAFAHNYDLKNHMRVHTGLRPYQCDSCCKTFVRSDH
LHRHLKKDGCNGVPSRRGRKPRVRGGAPDPSPGATATPGAPAQPSSPD
ARRNGQEKHFKDEDEDEDVASPDGLGRLNVAGAGGGGDSGGGPGAATD
GNFTAGLA

LRF-BTB/POZ domain: http://pfam.xfam.org/protein/ZBT7A_HUMAN

SEQ ID NO 2.:
IYRFVTRASVEERITQVAKRKMMLTHLVVRPGLGSKAGSMSKQELDDI
LKFGTEELFKDENEGENKEEDSSVIHYDNEAIARLLDRNQDATEDTDV
QNMNEYLSSFKAQYVVREEDKIEEIEREIIKQEENVDPDYWEKLLRHH
YEQQQEDLARNLGKGKRVRKQVNYNDAAQEDQDNQSEYSVGSEEEDED
FDERP

from Human CHD3:

MKAADTVILWARSKNDQLRISFPPGLCWGDRMPDKDDIRLLPSALGVK
KRKRGPKKQKENKPGKPRKRKKRDSEEEFGSERDEYREKSESGGSEYG
TGPGRKRRRKHREKKEKKTKRRKKGEGDGGQKQVEQKSSATLLLTWGL
EDVEHVFSEEDYHTLTNYKAFSQFMRPLIAKKNPKIPMSKMMTILGAK
WREFSANNPFKGSAAAVAAAAAAAAAAVAEQVSAAVSSATPIAPSGPP
ALPPPPAADIQPPPIRRAKTKEGKGPGHKRRSKSPRVPDGRKKLRGKK
MAPLKIKLGLLGGKRKKGGSYVFQSDEGPEPEAEESDLDSGSVHSASG
RPDGPVRTKKLKRGRPGRKKKKVLGCPAVAGEEEVDGYETDHQDYCEV
CQQGGEIILCDTCPRAYHLVCLDPELDRAPEGKWSCPHCEKEGVQWEA
KEEEEEYEEEGEEEGEKEEEDDHMEYCRVCKDGGELLCCDACISSYHI
HCLNPPLPDIPNGEWLCPRCTCPVLKGRCQKILHWRWGEPPVAVPAPQ
QADGNPDVPPPRPLQGRSEREFFVKWVGLSYWHCSWAKELQLEIFHLV
MYRNYQRKNDMDEPPPLDYGSGEDDGKSDKRKVKDPHYAEMEEKYYRF
GIKPEWMTVHRIINHSVDKKGNYHYLVKWRDLPYDQSTWEEDEMNIPE
YEEHKQSYWRHRELIMGEDPAQPRKYKKKKKELQGDGPPSSPTNDPTV
KYETQPRFITATGGTLHMYQLEGLNWLRFSWAQGTDTILADEMGLGKT
IQTIVFLYSLYKEGHTKGPFLVSAPLSTIINWEREFQMWAPKFYVVTY
TGDKDSRAIIRENEFSFEDNAIKGGKKAFKMKREAQVKFHVLLTSYEL
ITIDQAALGSIRWACLVVDEAHRLKNNQSKFFRVLNGYKIDHKLLLTG
TPLQNNLEELFHLLNFLTPERFNNLEGFLEEFADISKEDQIKKLHDLL
GPHMLRRLKADVFKNMPAKTELIVRVELSPMQKKYYKYILTRNFEALN
SRGGGNQVSLLNIMMDLKKCCNHPYLFPVAAMESPKLPSGAYEGGALI
KSSGKLMLLQKMLRKLKEQGHRVLIFSQMTKMLDLLEDFLDYEGYKYE
RIDGGITGALRQEAIDRFNAPGAQQFCFLLSTRAGGLGINLATADTVI
IFDSDWNPHNDIQAFSRAHRIGQANKVMIYRFVTRASVEERITQVAKR
KMMLTHLVVRPGLGSKAGSMSKQELDDILKFGTEELFKDENEGENKEE
DSSVIHYDNEAIARLLDRNQDATEDTDVQNMNEYLSSFKVAQYVVREE
DKIEEIEREIIKQEENVDPDYWEKLLRHHYEQQQEDLARNLGKGKRVR
KQVNYNDAAQEDQDNQSEYSVGSEEEDEDFDERPEGRRQSKRQLRNEK
DKPLPPLLARVGGNIEVLGFNTRQRKAFLNAVMRWGMPPQDAFTTQWL
VRDLRGKTEKEFKAYVSLFMRHLCEPGADGSETFADGVPREGLSRQQV
LTRIGVMSLVKKKVQEFEHINGRWSMPELMPDPSADSKRSSRASSPTK
TSPTTPEASATNSPCTSKPATPAPSEKGEGIRTPLEKEEAENQEEKPE
KNSRIGEKMETEADAPSPAPSLGERLEPRKIPLEDEVPGVPGEMEPEP
GYRGDREKSATESTPGERGEEKPLDGQEHRERPEGETGDLGKREDVKG
DRELRPGPRDEPRSNGRREEKTEKPRFMFNIADGGFTELHTLWQNEER
AAISSGKLNEIWHRRHDYWLLAGIVLHGYARWQDIQNDAQFAIINEPF
KTEANKGNFLEMKNKFLARRFKLLEQALVIEEQLRRAAYLNLSQEPAH
PAMALHARFAEAECLAESHQHLSKESLAGNKPANAVLHKVLNQLEELL
SDMKADVTRLPATLSRIPPIAARLQMSERSILSRLASKGTEPHPTPAY
PPGPYATPPGYGAAFSAAPVGALAAAGANYSQMPAGSFITAATNGPPV
LVKKEKEMVGALVSDGLDRKEPRAGEVICIDD

LRF-BTB binding region to CHD3 represented in bold

DUF1087: http://pfam.xfam.org/family/PF06465 represented in underlined portions of sequence.

SEQ ID NO. 3:
IYRFVTRASVEERITQVAKKKMMLTHLVVRPGLGSKTGSMSKQELDDI
LKFGTEELFKDEATDGGGDNKEGEDSSVIHYDDKAIERLLDRNQDETE
DTELQGMNEYLSSFKVAQYVVREEEMGEEEEVEREIIKQEESVDPDYW
EKLLRHHYEQQQEDLARNLGKGKRIRKQVNYNDGSQEDRDWQDDQSDN
QSDYSVASEEGDEDFDERS

from Human CHD4:

MASGLGSPSPCSAGSEEEDMDALLNNSLPPPHPENEEDPEEDLSETET
PKLKKKKKPKKPRDPKIPKSKRQKKERMLLCRQLGDSSGEGPEFVEEE
EEVALRSDSEGSDYTPGKKKKKKLGPKKEKKSKSKRKEEEEEEDDDDD
SKEPKSSAQLLEDWGMEDIDHVFSEEDYRTLTNYKAFSQFVRPLIAAK
NPKIAVSKMMMVLGAKWREFSTNNPFKGSSGASVAAAAAAAVAWESMV
TATEVAPPPPPVEVPIRKAKTKEGKGPNARRKPKGSPRVPDAKKPKPK
KVAPLKIKLGGFGSKRKRSSSEDDDLDVESDFDDASINSYSVSDGSTS
RSSRSRKKLRTTKKKKKKGEEEVTAVDGYETDHQDYCEVCQQGGEIDL
CDTCPRAYHMVCLDPDMEKAPEGKWSCPHCEKEGIQWEAKEDNSEGEE
ELEEVGGDLEEEDDHHMEFCRVCKDGGELLCCDTCPSSYHIHCLNPPL
PEIPNGEWLCPRCTCPALKGKVQKILIWKWGQPPSPTPVPRPPDADPN
TPSPKPLEGRPERQFFVKWQGMSYWHCSWVSELQLELHCQVMFRNYQR
KNDMDEPPSGDFGGDEEKSRKRKNKDPKFAEMEERFYRYGIKPEWMMI
HRILNHSVDKKGHVHYLIKWRDLPYDQASWESEDVEIQDYDLFKQSYW
NHRELMRGEEGRPGKKLKKVKLRKLERPPETPTVDPTVKYERQPEYLD
ATGGTLHPYQMEGLNWLRFSWAQGTDTILADEMGLGKTVQTAVFLYSL
YKEGHSKGPFLVSAPLSTIINWEREFEMWAPDMYVVTYVGDKDSRAII
RENEFSFEDNATRGGKKASRMKKEASVKFHVLLTSYELITIDMAILGS
IDWACLIVDEAHRLKNNQSKFFRVLNGYSLQHKLLLTGTPLQNNLEEL
FHLLNFLTPERFHNLEGFLEEFADIAKEDQIKKLHDMLGPHMLRRLKA
DVFKNMPSKTELIVRVELSPMQKKYYKYILTRNFEALNARGGGNQVSL
LNVVMDLKKCCNHPYLFPVAAMEAPKMPNGMYDGSALIRASGKLLLLQ
KMLKNLKEGGHRVLIFSQMTKMLDLLEDFLEHEGYKYERIDGGITGNM
RQEAIDRFNAPGAQQFCFLLSTRAGGLGINLATADTVIIYDSDWNPHN
DIQAFSRAHRIGQNKKVMIYRFVTRASVEERITQVAKKKMMLTHLVVR
PGLGSKTGSMSKQELDDILKFGTEELFKDEATDGGGDNKEGEDSSVIH
YDDKAIERLLDRNQDETEDTELQGMNEYLSSFKVAQYVVREEEMGEEE
EVEREIIKQEESVDPDYWEKLLRHHYEQQQEPLARNLGKGKRIRKQVN
YNPGSQEDRDWQDDQSDNQSDYSVASEEGPEDFDERSEAPRRPSRKGL
RNDKDKPLPPLLARVGGNIEVLGFNARQRKAFLNAEVIRYGMPPQDAF
TTQWLVRDLRGKSEKEFKAYVSLFMRHLCEPGADGAETFADGWREGLS
RQHVLTRIGVMSLIRKKVQEFEHVNGRWSMPELAEVEEMKKMSQPGSP
SPKTPTPSTPGDTQPNTPAPVPPAEDGDCIEENSLKEEESIEGEKEVK
STAPETAIECTQAPAPASEDEKVWEPPEGEEKVEKAEVKERTEEPMET
EPKGAADVEKVEEKSAIDLTPIVVEDKEEKKEEEEKKEVMLQNGETPK
DLNDEKQKKNIKQRFMFNIADGGFTELHSLWQNEERAATVTKKTYEIW
HRRHDYWLLAGIINHGYARWQDIQNDPRYAILNEPFKGEMNRGNFLEI
KNKFLARRFKLLEQALVIEEQLRRAAYLNMSEDPSHPSMALNTRFAEV
ECLAESHQHLSKESMAGNKPANAVLHKVLKQLEELLSDMKADVTRLPA
TIARIPPVAVRLQMSERNILSRLANRAPEPTPQQVAQQQ

LRF-BTB binding region to CHD4 represented in bold

DUF1087: http://pfam.xfam.org/family/PF06465 represented in underlined portions of sequence.

Unbiased Approach: Protein-Fragment Complementation Assay (PCA)

Applicants employ PCA to perform a cell-based, high-throughput screen (HTS) for small molecules which interferes with the binding between the LRF-BTB domain and CHD3-LBD (Masuda et. al. Science in press). PCA is a useful tool to monitor the dynamics of protein-protein interactions in vivo (Michnick et al. 2007).

In this strategy, cDNAs encoding two proteins of interest are fused to complementary fragments encoding a reporter protein. If the two proteins interact, the reporter fragments are brought together, fold into the native structure and reporter activity is reconstituted. Applicants previously used this assay to monitor the dynamics of LRF-BTB homodimer formation in cells (Sakurai N, Maeda M, Lee S U, Ishikawa Y, Li M, et al. The LRF transcription factor regulates mature B cell development and the germinal center response in mice. J Clin Invest. 2011; 121:2583-2598). Applicants chose the humanized form of G. princeps luciferase (hGLuc) as a reporter protein, since it can generate a greater than 100-fold higher bioluminescence signal than Photinus pyralis (firefly) luciferase or Renilla reniformis luciferase (R-Luc) (Tannous B A, Kim D E, Fernandez J L, Weissleder R., Breakefield X O. Codon-optimized Gaussia luciferase cDNA for mammalian gene expression in culture and in vivo. Mol Ther. 2005; 11:435-443). The 5′ (hGluc1) and 3′ (hGLuc2) sequences of the gene encoding hGluc were fused to human LRF-BTB coding sequences with nuclear localization sequences (NLS) and the resulting fusions (LRF-BTB-hGluc1 and LRF-BTB-hGLuc2) were co-expressed in HEK293 cells (Sakurai et al. 2011). GCN4 leucine zipper protein served as a positive control (Zip-hGLuc1 and Zip-hGLuc2) (Remy I, Michnick S W. A highly sensitive protein-protein interaction assay based on Gaussia luciferase. Nat Methods. 2006; 3:977-979). LRF-BTB induced complementation of hGLuc fragments reconstituting hGluc activity in a dose-dependent manner. Importantly, this reaction was LRF-BTB-specific, as the combination of two unbound proteins (Zip-hGLuc1 and LRF-BTB-hGLuc2) failed to reconstitute hGLuc activity.

Applicants utilize a HEK293 line that expresses CHD3-LBD-hGLuc1 and LRF-BTB-GLuc2 (293-LC) for a cell-based HTS. The 293-LC line exhibits high hGLuc reporter activity due to stable interaction between the two proteins (CHD3-LBD-hGLuc1 and LRF-BTB-GLuc2). The 293-LC line also expresses Renilla reniformis luciferase as an internal control to monitor cell viability. Applicants then screen small molecules (collection comprising 500,000 molecules) for those that reduce hGLuc expression.

Materials and Methods

Mice

Hematopoietic-specific conditional Zbtb7a knockout mice (Zbtb7a^{F/F} Mx1-Cre+) were described previously (6, 26). Zbtb7a^{F/F} Mx1-Cre+ mice were bred with the β-YAC mice (K. R. Peterson, C. H. Clegg, C. Huxley, B. M. Josephson, et al., Proceedings of the National Academy of Sciences 90, 7593-7 (1993)) to establish Zbtb7a conditional KO mice harboring the human β-globin gene cluster as a yeast artificial chromosome transgene. pIpC was injected intraperitoneally at 8 weeks of age, when human γ-globin expression is silenced in this model (J. Xu, C. Peng, V. G. Sankaran, Z. Shao, et al., Science 334, 993-6 (2011)).

Cell Sorting

To collect splenic erythroblasts for RNA-Seq, mice were treated with phenylhydrazine and splenocytes harvested as described (Y. Ishikawa, M. Maeda, M. Pasham, F. Aguet, et al., Haematologica 100, 439-51 (2015)). After preparing single-cell suspensions using a cell strainer (BD), splenocytes were incubated with red cell lysis buffer and then incubated with a mixture of biotin-conjugated antibodies (anti-CD11b, Gr-1, CD19, B220, CD3, CD4 and CD8), followed by incubation with anti-biotin MicroBeads (Miltenyi Biotec). Cell suspensions were subsequently applied onto MACS separation LD columns (Miltenyi Biotec) for negative selection. Cells were then incubated with fluorochrome-conjugated anti-CD71, -TER119 and -CD44 antibodies and subjected to sorting. Cell sorting was performed at the Children's Hospital Boston Flow Cytometry Core Facility (CHBFCC) with an AriaIII cytometer using DAPI for live/dead discrimination.

Primary Human Erythroid Culture

G-CSF-mobilized adult human PB CD34⁺ cells were obtained from the Hematopoietic Cell Processing Core at Fred Hutchinson Cancer Center, and erythroid differentiation induced as described with minor modifications (13, 22). The erythroid culture system consists of three phases using three different erythroid differentiation media (EDM-I, -II, and -III) (FIG. 7A). The composition of basal EDM is: IMDM (Cellgro) supplemented with 1% L-glutamine (Life Technologies), 2% penicillin-streptomycin (Life Technologies), holo-human transferrin (330 μg/ml, Sigma), heparin (2 IU/mL, Sigma), recombinant human insulin (10 μg/ml, Sigma), erythropoietin (3 IU/mL, Amgen) and 5% inactivated human plasma (Rhode Island Blood Center). After thawing, cells were resuspended in EDM-I [EDM supplemented with hydrocortisone (10⁻⁶M, Sigma), hSCF (100 ng/ml, R&D) and hIL3 (5 ng/ml, R&D)] and cultured for 7 days (phase I). On day 7, medium was replaced with EDM-II [EDM supplemented with hSCF (100 ng/ml)] (phase II) and, on day 11 medium was switched to EDM-III (EDM with no supplement) (phase III).

### HUDEP-2 Cells

HUDEP-2 cells were maintained with StemSpan STEM medium (Stem Cell Technologies) supplemented with hSCF (50 ng/mL), erythropoietin (3 IU/ml), dexamethasone (10⁻⁶M, Sigma) and doxycycline (dox: 1 μg/mL, Sigma) (Kurita 2013). To induce differentiation, medium was replaced with EDM-II and cells were cultured for 5 days in the presence of dox (FIG. 8A). If necessary, cells were cultured for 2 more days without dox and used for experiments (e.g. HPLC, Hb-FACS).

Generation of ZBTB7A^{Δ/Δ}, BCL11A^{Δ/Δ} and ZBTB7A^{Δ/Δ}BCL11A^{Δ/Δ}HUDEP-2 Cells

We first established a HUDEP-2 line stably expressing Cas9 endonuclease (HUDEP-2_Cas9) using a lentivirus vector encoding Cas9 and a Blasticidin-resistance cassette (lentiCas9-Blast; Addgene plastnid ID 52962). To generate ZBTB7A or BCL11A KO HUDEP-2 cells, sgRNA sequences (ATCGGGATCCCGTFCCCCGA or TGAACCAGACCACGGCCCGT) were designed to target ZBTB7A or BCL11A, respectively (L. Cong, F. A. Ran, D. Cox, S. Lin, et al., Science 339, 819-23 (2013)). sgRNA-specifying oligos were subcloned into the lentiGuide-Puro vector (Addgene plasmid ID 52963). Lentivirus transduction was performed as previously described (Ishikawa 2015). Puromycin- and blasticidin-resistant cells were harvested 2 weeks after transduction and knockout was confirmed by Western blot. Independent LRF (or BCL11A) KO clones were established via limiting dilution as described (M. C. Canver, D. E. Bauer, A. Dass, Y. Y. Yien, et al., J Biol Chem (2014)). DKO HUDEP-2 cells (ZBTB7A^{Δ/Δ}BCL11A^{Δ/Δ}) were generated by targeting the BCL11A gene in ZBTB7A^{Δ/Δ} HUDEP-2 cells.

Hemoglobin FACS

Cells were fixed with 4% paraformaldehyde at room temperature (RT) for 20 min, washed with PBS and permeabilized with 0.1% Triton X-100 (for 10 min at RT). After a PBS wash, cells were first incubated with primary antibody (anti-HbF or non-specific IgG) for 30 min, washed with PBS and then incubated with PE/Cy7-conjugated secondary antibody for 30 min. Incubation with antibodies was performed at RT on a rocking shaker. Antibodies used were: anti-Human Fetal Hemoglobin (2D12, BD), non-specific mouse IgG1κ (555748, BD) and PE/Cy7-conjugated anti-mouse IgG1 antibody (406613, Biolegend).

Hemoglobin HPLC

Three to five million HSPC-derived erythroblasts or HUDEP cells were spun down, and cell pellets were subjected to HPLC. Over the course of this study, we measured HbF levels using a G7 HPLC Analyzer (TOSOH BIOSCIENCE, INC) in two different modes (hemoglobin A1c or beta-thalassemia programs). To compare HbF levels across experiments (performed in different modes), we defined % HbF as a proportion of HbF relative to HbA0.

Antibodies for FACS

Antibodies were purchased from Biolegend, eBioscience, BD or Mittenyi Biotec. Fluorochrome-conjugated antibodies included: TER119 (TER-119), mouse CD44 (IM7), mouse CD71 (R17217), human CD235a (HIR2), human CD71 (CY1G4), human CD36 (5-271) and human α4-integrin (MZ18-24A9). The following biotin-conjugated antibodies were used for negative selection: Biotin-CD11b (M1/70), Biotin-Gr1 (RB6-8C5), Biotin-B220 (RA3-6B2), Biotin-CD19 (eBio1D3), Biotin-CD3 (145-2C11), Biotin-CD4 (L3T4) and Biotin-CD8 (eBio-H35-17.2).

Isoelectric Focusing of PB Hemolysates and MALDI-TOFMS Analysis

Hemoglobin was analyzed by isoelectric focusing (IEF) (Resolve; PerkinElmer) as per manufacturer's instructions running for 45 minutes at 300 volts at 15° C. (J. Black, Hemoglobin 8, 117-27 (1984)). Focused hemoglobins were excised from the IEF gel, digested with trypsin (A. Shevchenko, H. Tomas, J. Havlis, J. V. Olsen, M. Mann, Nat Protoc 1, 2856-60 (2006)) and analyzed using a matrix-assisted laser desorption/ionization time-of flight (MALDI-TOF/TOF) mass spectrometer (Ultralex Bruker Daltonics) (H. B. Kramer, K. J. Lavender, L. Qin, A. R. Stacey, et al., PLoS Pathog 6, e1000893 (2010)).

Western Blot Analysis

Western blot analysis was performed using standard methodology with the following antibodies: LRF (13E9, eBioscience), BCL11A (ab19487, Abcam), GATAD2B (A301-283, Bethyl), MTA2 (ab8106, Abcam), HDAC1 (ab19845, Abcam), HDAC2 (ab32117, Abcam), GAPDH (sc-25778, Santa Cruz Biotechnology), HSP90 (clone: 68, BD), αTubulin (DM1A, Sigma), HBG1/2 (Hemoglobin γ: sc-21756, Santa Cruz Biotechnology) and Flag (M2, Sigma).

Wright-Giemsa Stain

Wright-Giemsa staining was performed using PROTOCOL Wright-Giemsa Stain Solutions (Fisher Scientific) following the manufacturer's specifications.

Plasmids

Lentivirus vectors expressing shRNA-against human ZBTB7A (pLKO.1-shRNA vectors) were purchased from Sigma. Two independent shRNA clones were used: TRCN0000137891 (ZBTB7A clone #2) and TRCN0000136851 (ZBTB7A clone #4).

The following plasmids were used for Co-IP experiments: pCMV-Tag2B-Flag-p66beta (human GATAD2B/p66β cDNA was obtained from the pcDNA3-mCherry-p66beta vector from Dr. Rainer Renkawitz), pcDNA3 N-Flag-CHD8 (from Dr. Keiichi Nakayama), pCI-neo3-Flag-hCHD3 (from Dr. Aaron Goodarzi), pcDNA3.1-hACF1-Flag (from Dr. Patrick Varga-Weisz) and pEF1-hLRF-V5His-Neo. For immunoprecipitation in MEL cells, we used the pMX-mCherry-FHC-mLRF retrovirus vector, in which Flag- and HA-tags were introduced at the 3′ end of mouse LRF cDNA (FIG. 15C).

Real Time PCR Assay

RNA was extracted using TRIZOL (Life Technologies) and treated with DNase I (Life Technologies). cDNA was synthesized using the High-Capacity cDNA Reverse Transcription Kit (ABI) and Real-time PCR was performed using Fast EvaGreen qPCR Master Mix (Biotium) and a ABI7900HT system (ABI). Transcript levels were calculated relative to corresponding β-actin (mouse) or RPS18 (human) levels in each sample. Primer sequences for qRT-PCR were:

mouse β-actin FW:
5′-ACCCTAAGGCCAACCGTGA-3′,
mouse β-actin RV:
5′-GTCTCCGGAGTCCATCACAA-3′,
mouse Hbb-bh1 FW:
5′-CTCAAGGAGACCTTTGCTCA-3′,
mouse Hbb-bh1 RV:
5′-AATCACCAGCTTCTGCCAGGC-3′,
mouse Hbb-bs/bt FW:
5′-TTTAACGATGGCCTGAATCACTT-3′,
mouse Hbb-bs/bt RV:
5′-CAGCACAATCACGATCATATTGC-3′,
human HBB FW:
5′-AGGAGAAGTCTGCCGTTACTG-3′,
human HBB RV:
5′-CCGAGCACTTTCTTGCCATGA-3′,
human HBG1/2 FW:
5′-AACCCCAAAGTCAAGGCACA-3′,
human HBG1/2 RV:
5′-CATCTTCTGCCAGGAAGCCT-3′,
human HBE1 FW:
5′-GAGAGGCAGCAGCACATATC-3′,
human HBE1 RV:
5′-CAGGGGTAAACAACGAGGAG-3′,
human RPS18 FW:
5′-GTAACCCGTTGAACCCCATT-3′,
human RPS18 RV:
5′-CCATCCAATCGGTAGTAGCG-3′.

Retrovirus Transduction

To transduce MEL cells, 293T cells were transfected with the pMX-mCherry-FHC-mLRF vector and the pMCV-Ecopac vector using Lipofectamine 2000, and virus-containing supernatants were collected. 2×10⁵ MEL cells were spin-infected with 1 ml viral supernatant containing polybrene (5 μg/mL) and then selected in puromycin (2 μg/mL) starting 48 hours later.

Generation and Sequencing of RNA-Seq Libraries

Total RNA was extracted from FACS-sorted splenic erythroblasts (or HUDEP-2 cells) using RNeasy Mini Kit (QIAGEN). 125 ng of purified RNA was used for library construction. Libraries were generated using the Encore Complete RNA-Seq DR Multiplex System following the manufacturer's specifications (Nugen). All libraries underwent 50 bp paired-end sequencing on an Illumina HiSeq 2000 or 2500.

RNA-Seq Data Analysis

Sequenced reads were aligned to the UCSC hg19 (human, mm9 mouse) annotation using tophat2 aligner, and read counts were quantified using HTSeq (S. Anders, P. T. Pyl, W. Huber, Bioinformatics 31, 166-9 (2015)). Differential expression analysis was carried out using DESeq Bioconductor package (S. Anders, W. Huber, Genuine Biol 11, R106 (2010)). Hba-a1/Hba-a2 and Hbb-b1/Hbb-b2 reads were pooled during the alignment stage to estimate their combined expression magnitude. GSEA of differentially expressed genes was carried out using log₂ fold-change values, restricting the gene set to those observed with an FPM (Fragments Per Million mapped reads) value of >1.0 in at least one condition, using a power factor of 1.

Generation and Sequencing of ChIP-Seq Libraries

LRF-ChIP-Seq experiments were performed using HSPC-derived erythroblasts (Day 8 erythroblasts) in duplicate and undifferentiated HUDEP-2 cells in triplicate. Five million cells were fixed with 1% formaldehyde at RT for 20 min and chromatin was collected using a truChIP High Cell Chromatin Shearing Kit with SDS (Covaris). Sonication was performed using a Covaris S2 instrument (Covaris). The chromatin solution was first incubated with 40 μl protein A/G Dynabeads (Life Technologies) at 4° C. for 3 h on a rotating shaker to prevent non-specific binding. ChIP was then performed using 1 μg anti-LRF antibody (clone13E9, eBioscience) at 4° C. overnight on a rotating shaker. The antibody/protein complex was harvested following incubation with 15 μL of BSA-blocked protein A/G Dynabeads at 4° C. for 1 h. Beads were sequentially washed once with the following buffers: wash buffer A (10 mM Tris-HCl, pH7.4, 1 mM EDTA, 1% Triton X-100, 0.1% SDS, 0.1% SDC and 1 mM DTT), wash buffer B (10 mM Tris-HCl, pH7.4, 1 mM EDTA, 1% Triton X-100, 0.1% SDS, 0.1% SDC, 300 mM NaCl and 1 mM DTT), wash buffer C (250 mM LiCl, 0.5% NP40 and 0.5% SDC) and finally TE buffer. Complexes were then eluted from beads in 50 mM Tris-HCl (pH8.0)/1% SDS/10 mM EDTA. After reverse cross-linking (at 65° C. overnight) and proteinase K treatment, DNA was extracted using a QIAquick PCR purification kit (QIAGEN). ChIP-Seq libraries were generated using a Ovation Ultralow DR Multiplex System 1-8 (NuGEN) and sequenced. All libraries were sequenced by 50 bp single-end or paired-end reads using an Illumina HiSeq 2000 or 2500 system.

ChIP-Seq Data Analysis

ChIP-Seq reads were aligned (hg18 assembly for human, mm9 assembly for mouse) using bowtie2. Smoothed read density, enrichment profiles and peak calls were generated using the spp package (P. V. Kharchenko, M. Y. Tolstorukov, P. J. Park, Nat Biotechnol 26, 1351-9 (2008)). A consistent set of LRF binding positions was determined using IDR procedure (IDR=0.05). Sequence motifs were determined using the MEME package (JASPAR_CORE and uniprobe motifs) using +/−100 bp regions around the op 500 sites. To better resolve LRF enrichment within the γ-globin locus, we performed paired-end alignment and estimated conservative enrichment profiles (lower bound of 95% confidence interval; see spp package (Kharchenko 2008)) using i) all mappable fragments and ii) only uniquely-mappable fragments (bottom tracks, FIG. 4).

Generation and Sequencing of ATAC-Seq Libraries

ATAC-Seq libraries were constructed using 7.5×10⁴ HUDEP-2 cells per sample as described (18, 36). After a transposition reaction, transposed DNA fragments were purified using a MinElute Kit (Qiagen) and PCR-amplified using PCR primer1 (Ad1_noMX) and a barcoded PCR primer2 (Ad2) (18, 36). PCR conditions used were: 72° C. for 5 min, 98° C. for 30 s, followed by 11 cycles of 98° C. for 10 s, 63° C. for 30 s and 72° C. for 1 min. Amplified libraries were purified with a MinElute Kit and library quality was assessed using the TapeStation system (Agilent). All libraries were sequenced by 50 bp paired-end reads using the illumina Hi(63Seq 2500 system.

ATAC-Seq Data Analysis

Sequenced reads were aligned using bowtie2, an PCR duplicates were removed using the “samtools rmdup” command. Aligned fragments with length <156 bp were used to calculate the hypersensitivity profile: smoothed density of fragment ends was calculated using the spp package with the a bandwidth of 100 bp. Peaks of smoothed read density greater than 1 read per million were used to estimate position of hypersensitivity sites. Statistically significant ATAC-seq differences between LRF KO and WT HUDEP-2 cells (red tracks in FIG. 4) were evaluated using conservative enrichment estimates (lower bound of the 95% confidence interval) in the spp package (P. V. Kharchenko, M. Y. Tolstowkov, P. J. Park, Nat Biotechnol 26, 1351-9 (2008))with a 500 bp window size.

Yeast Two-Hybrid Screen (Y2H)

A Y2H screen (ULTimate Y2H™, Hybrigenics) was performed with the human LRF-BTB domain (aa 1-131) as bait using a cDNA library constructed from human B-cell lymphoma cell lines (SUDHL-4, SUDIIL-6 and RCK8). The human LRF-BTB domain coding sequence (aa 1-131) was PCR-amplified and subcloned into the pB27 (N-LexA-bait-C fusion) vector (Hybrigenics). The interaction assay uses a His3 reporter that allows yeast to grow in medium lacking histidine A. B. Vojtek, S. M. Hollenberg, J. A. Cooper, Cell 74, 205-14 (1993)). Autoactivation of the bait fragment was tested in the presence of 3-aminotriazole (3-AT). A total of 52.1 million interactions were analyzed and 360 positive clones processed for analysis.

Immunoprecipitation

Immunoprecipitation was performed using nuclear protein extracts of HSPC-derived erythroblasts (day 8) or MEL cells. Nuclei were isolated by incubating cell pellets (3×10⁷) in lysis buffer A (10 mM HEPES-NaOH, pH7.9, 10 mM KCl, 1.5 mM MgCl2 and 1 mM DTT) for 10 min on ice. During incubation, the solution was mixed by 10 strokes of gentle pipetting. Nuclear pellets were fixed with 3% formaldehyde (30 min at RT), washed with PBS 4 times, and incubated 10 min on ice in lysis buffer B (10 mM HEPES-NaOH, pH7.9, 0.2% SDS, 0.1% SLS, 2 mM EDTA, 1 mM EGTA and 50 mM NaCl). After brief sonication (Bioruptor, Diagenode), nuclear extracts were collected and subjected to immunoprecipition using the following antibodies: LRF (clone 13E9, eBioscience), GATAD2B (A301-282, Bethyl) and MTA2 (ab8106, Abcam).

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A method of treating or inhibiting progression of hemoglobinopathy in a subject in need thereof comprising inhibiting interaction between LRF-BTB and CHD protein-LBD.

2. The method of claim 1 wherein the hemoglobinopathy comprises sickle cell anemia, or β-thalassemia.

3. The method of claim 1 comprising administering to the subject an effective amount of a small molecule, peptide, or antibody that inhibits the interaction in order to induce HbF expression.

4. The method of claim 3 wherein the antibody binds to LRF-BTB and/or CHD protein-LBD and/or CHD protein-domain and LRF-BTB when interacting.

5. The method of claim 1 wherein the CHD comprises CHD3.

6. The method of claim 1 wherein the CHD comprises CHD4.

7. A method for identifying an agent as a potential inhibitor of LRF-BTB/CHD protein-LBD interaction for treating hemoglobinopathy comprising:
(a) generating a series of N-terminal and C-terminal deletion mutations of the CHD protein-LBD consisting of an amino acid sequence having at least 95% identity with an amino acid sequence for CHD protein-LBD having SEQ ID NO. 2 or SEQ ID NO. 3, wherein the N-terminal and C-terminal deletion mutations are fragments;
(b) measuring the binding of the fragments to the LRF-BTB domain;
(c) comparing the interaction of the fragments to the LRF-BTB domain to a control to determine the relative strength of the binding between the fragments and the CHD protein CHD3/LRF-BTB domain or the CHD protein CHD4/LRF-BTB domain.

8. The method for identifying an agent as a potential inhibitor according to claim 7, wherein the relative strength is measured by surface plasmon resonance.

9. A method of identifying an agent as a potential inhibitor of LRF-BTB/CHD protein CHD3-LBD interaction for treating hemoglobinopathy comprising:
(a) co-introducing a first nucleic acid construct and a second nucleic acid construct into a cell or cell population,
(b) wherein the first nucleic acid construct comprises a nucleotide sequence which codes for a fusion protein which comprises an LRF-BTB domain linked to a reporter protein, wherein the LRF-BTB domain comprises an amino acid sequence having at least 95% identity with SEQ ID NO. 1, and the second nucleic acid construct comprises a nucleotide sequence which codes for a second fusion protein which comprises an CHD domain linked to a second reporter protein, wherein the CHD domain comprises an amino acid sequence for CHD3 or CHD4 having at least 95% identity with SEQ ID NO. 2 or 3;
(c) allowing the cell or cell population to express the first fusion protein and the second fusion protein and contacting the cell or cell population with a potential inhibitor of LRF-BTB/CHD protein-LBD interaction; and,
(d) detecting or quantifying an increase or decrease in protein complex formation, a change in subcellular localization, a concentration of signal or combination thereof, wherein a change in fluorescence indicates disruption of protein complex formation.

10. The method of claim 9, wherein the reporter protein is selected from the group consisting of a luciferase, a lactosidase, a green fluorescent protein, a yellow fluorescent protein, a cyan fluorescent protein and a red fluorescent protein.

11. The method of claim 10, wherein the reporter protein is a humanized form of G. princeps luciferase.

12. The method of claim 9, wherein the cell or cell population comprises HEK293 cells.

13. The method of claim 9, wherein the hemoglobinopathy comprises sickle cell anemia, or β-thalassemia.

14. (canceled)

15. The method of claim 9 comprising administering to the subject an effective amount of a small molecule, peptide, or antibody small molecule, peptide, or antibody.

16. The method of claim 9, wherein the method is performed as a medium- or high-throughput screening.

17. The method of claim 9, wherein the CHD domain comprises an amino acid sequence for CHD3.

18. The method of claim 9, wherein the CHD domain comprises an amino acid sequence for CHD4.

19. An inhibitor of LRF-BTB/CHD protein CHD3-LBD interaction identified by a method of claim 7.

20. The inhibitor of claim 19 comprising a small molecule, peptide, or antibody.

21. An inhibitor of LRF-BTB/CHD protein CHD3-LBD interaction comprising a small molecule, peptide, or antibody.

22. A method of determining a minimal amino acid sequence between an interaction of a CHD protein domain, wherein the CHD protein domain comprises an amino acid sequence having at least 95% identity with an amino acid sequence for CHD3 or CHD4 comprising SEQ ID NO. 2 or 3 and a LRF-BTB domain, wherein the LRF-BTB domain comprises an amino acid sequence having at least 95% identity with SEQ ID NO. 1, the method comprising:
(a) generating a series of N-terminal and C-terminal deletion fragments of the LRF-BTB domain;
(b) measuring the binding of the fragments to the CHD domain; and,
(c) determining the relative strength of the binding between the fragments and the CHD domain.

23. The method of claim 22 wherein the relative strength is measured by surface plasmon resonance.

24. A method for identifying de-repressed β-globin expression comprising:
(a) introducing a vector that encodes and expresses a CHD protein/LRF-BTB domain fragment fused with a protein transduction domain into a cell or cell population, and
(b) determining and/or measuring the γ-globin level of expression.

25. The method of claim 24 wherein the vector comprises a lentiviral vector.

26. The method of claim 24 wherein the cell or cell population comprises HUDEP-2 cells.

27. The method of claim 24 wherein the protein transduction domain (PTD) comprises a PTD derived from a lentiviral TAT.

28. The method of claim 27 wherein the PTD derived from a lentiviral TAT comprises a PTD derived from an HIV TAT.

29. The method of claim 24 wherein the CHD comprises a CHD3.

30. The method of claim 24 wherein the CHD comprises a CHD4.
